(19) United States
(12) Patent Application Publication
(10) Pub. No.: US 2022/0387489 A1
(43) Pub. Date: Dec. 8, 2022

(54) METHODS TO PREPARE V-T CELLS DERIVED EXOSOMES FOR TREATMENT OF EPSTEIN-BARR VIRUS-ASSOCIATED CANCERS

(71) Applicant: THE UNIVERSITY OF HONG KONG, Hong Kong (CN)

(72) Inventors: Wenwei TU, Hong Kong (CN); Yinping LIU, Hong Kong (CN); Xiwei WANG, Hong Kong (CN); Zheng XIANG, Hong Kong (CN)

(21) Appl. No.: 17/754,992

(22) PCT Filed: Aug. 31, 2020
(86) PCT No.: PCT/CN2020/112573

§ 371 Date: Apr. 18, 2022

Related U.S. Application Data
(60) Provisional application No. 62/916,430, filed on Oct. 17, 2019.

(51) Int. Cl. A61K 35/17; C12N 5/0783

(52) U.S. Cl. CPC A61K 35/17; C12N 5/0636

(57) ABSTRACT

Provided are exosomes derived from Vδ2-T cell (Vδ2-T-Exos) for killing or inhibiting EBV-infected cells. Further provided is a method for killing or inhibiting the growth of an EBV-infected cell, comprising contacting the EBV-infected cell with exosomes from Vδ2+ T cells in an amount effective to kill or inhibit the growth of the cell. Preferably, the EBV-infected cell is an EBV-infected cell that has become neoplastic, such as an EBV-infected neoplastic B-cell or an EBV-infected neoplastic epithelial cell. Further provided is a method for treating an EBV-induced cancer in a subject by administering to the subject a therapeutically effective amount of Vδ2-T-Exos. Vδ2-T-Exos can be derived from Vδ2-T cells obtained from the subject or from an allogeneic healthy individual. Methods for isolating Vδ2-T-Exos from Vδ2-T cells are also provided.

### BACKGROUND OF THE INVENTION

Epstein-Ban virus (EBV) persistently infects most adults in an asymptomatic manner; however, it is also associated with a variety of lymphoid cancers. In immunocompromised patients, it may cause life-threatening EBV-induced B-cell lymphoproliferative disorders (EBV-LPD) and diffuse large B cell lymphoma. Current treatment options for EBV-associated tumors are very limited with remarkable unwanted off-target toxicities and incompletely effective for relapsed or refractory diseases. Restoration of immunity to EBV by adoptive transfer of ex vivo-generated EBV-specific cytotoxic T cells (CTL) was successful for treating an EBV-associated tumors in some hematopoietic-cell transplantation patients. However, it is ineffective for EBV-associated tumors in patients with solid-organ transplantation, and also limited by the difficulties in generating enough numbers of EBV-specific CTLs in vitro.

γδ-T cells, as the innate-like T cells with MHC-unrestricted lytic activities against different tumor cells, have a great potential in cancer immunotherapy. Human γδ-T cells are divided into two major subsets upon the incorporation of Vδ1 or Vδ2 chain in their T cell receptors (TCR). Vδ1⁺ T cells are dominant in mucosal and epithelial tissue, while most Vδ2⁺ T cells exist in the peripheral blood and lymphoid organs, and generally co-express Vγ9. Vδ2⁺ T cells can be activated and expanded in a MHC-independent manner by phosphoantigens, the small nonpeptidic phosphorylated intermediates of mevalonate pathway in mammalian cells. Pamidronate (PAM), a pharmacological aminobisphosphonate commonly used for the treatment of osteoporosis, can also selectively activate and expand human Vδ2⁺ T cells in vitro and in vivo. Recently, using immunodeficient Rag2^{-/-}γc^{-/-}, and humanized mouse EBV-LPD model, it was shown that either adoptive transfer of ex vivo PAM-expanded Vδ2⁺ T cells or direct administration of PAM to expand Vδ2⁺ T cells in vivo could control EBV-LPD, suggesting Vδ2-T-cell-based immunotherapy could be used to treat EBV-induced B-cell cancers. However, its clinical application is limited because Vδ2-T cells from some cancer patients are difficult to be expanded by phosphoantigens and repeated administration of phosphoantigens may result in Vδ2-T cell exhaustion. In addition, the antitumor efficacy of cell-based immunotherapy may be seriously impeded due to the immunosuppressive tumor microenvironment in patients.

### BRIEF SUMMARY OF THE INVENTION

This disclosure provides that human Vδ2-T cells-derived exosomes (Vδ2-T-Exos) not only directly kill EBV-induced B-cell lymphomas, but also indirectly inhibit lymphoma development and progression by enhancing T cell-mediated antitumor activities. Accordingly, certain embodiments of the invention provide a method of killing or inhibiting the growth of an EBV-infected cell by contacting the cell with Vδ2-T-Exos. Further embodiments of the invention also provide methods of treating an EBV-induced cancer, such as EBV-induced B-cell lymphoma, by administering to a subject Vδ2-T-Exos. Even further embodiments of the invention provide methods of isolating Vδ2-T-Exos.

### BRIEF DESCRIPTION OF THE DRAWINGS

FIG. 1A. Characterization of Vδ2-T-Exos. Size distribution of Vδ2-T-Exos measured by dynamic light scattering analysis.

FIG. 1B. Characterization of Vδ2-T-Exos. Morphology of Vδ2-T-Exos determined by transmission electron microscopy. Scale bar, 50 nm.

FIG. 1C. Characterization of Vδ2-T-Exos. Exosomal markers of CD63, TSG101, CD81, Alix and endoplasmic reticulum marker GRP94 in Vδ2-T cells and Vδ2-T-Exos were measured by western blot analysis.

FIG. 1D. Characterization of Vδ2-T-Exos. Surface expression of functional molecules on Vδ2-T-Exos determined by flow cytometry, the gray histograms represent isotype controls. Each experiment was conducted four times independently.

FIG. 1E. Characterization of Vδ2-T-Exos. Surface expression of functional molecules on Vδ2-T-Exos determined by flow cytometry, the gray histograms represent isotype controls. Each experiment was conducted four times independently.

FIG. 1F. Characterization of Vδ2-T-Exos. Surface expression of functional molecules on Vδ2-T-Exos determined by flow cytometry, the gray histograms represent isotype controls. Each experiment was conducted four times independently.

FIG. 2A. Vδ2-T-Exos target EBV-induced B-cell lymphoma. Vδ2-T-Exos were labeled with Dil and then cultured with EBV-LCL. After 18 h, Dil signal in EBV-LCL was analyzed by confocal microscopy. DAPI was used to stain nuclei (Dil=red, DAPI=blue; scale bar=10 μm). *p<0.05, **p<0.01. NS, not significant.

FIG. 2B. Vδ2-T-Exos target EBV-induced B-cell lymphoma. DiR-labeled Vδ2-T-Exos were injected i.p. into EBV-induced B-cell lymphomas bearing mice. Tumor tissues were harvested 3, or 24 hours later. The fluorescence density of DiR-labeled Vδ2-T-Exos was determined using an in vivo imaging system. *p<0.05, **p<0.01. NS, not significant.

FIG. 2C. Vδ2-T-Exos target EBV-induced B-cell lymphoma. CFSE-labeled Vδ2-T-Exos were cultured with EBV-LCL and autologous normal B cells for 18 h. CFSE signal in the cells were detected by flow cytometry. *p<0.05, **p<0.01. NS, not significant.

FIG. 2D. Vδ2-T-Exos target EBV-induced B-cell lymphoma. CFSE-labeled Vδ2-T-liposomes were cultured with EBV-LCL and autologous normal B cells for 18 h. CFSE signal in the cells were detected by flow cytometry. *p<0.05, **p<0.01. NS, not significant.

FIG. 2E. Vδ2-T-Exos target EBV-induced B-cell lymphoma. CFSE-labeled Vδ2-T-Exos were pre-incubated with neutralizing anti-NKG2D antibodies or isotype control and then subjected to culture with EBV-LCL. CFSE signal on EBV-LCL were determined after 18 h. Pellets isolated from non-conditioned Exos-free medium without Vδ2-T cell components were served as control. All the data shown as mean±SEM are representative of four independent experiments. *p<0.05, **p<0.01. NS, not significant.

FIG. 2F. Vδ2-T-Exos target EBV-induced B-cell lymphoma. CFSE-labeled Vδ2-T-Exos were pre-incubated with neutralizing anti-TCR-γδ antibodies or isotype control and then subjected to culture with EBV-LCL. CFSE signal on EBV-LCL were determined after 18 h. Pellets isolated from non-conditioned Exos-free medium without Vδ2-T cell components were served as control. All the data shown as mean±SEM are representative of four independent experiments. *p<0.05, **p<0.01. NS, not significant.

FIG. 3A. Vδ2-T-Exos induce apoptosis of EBV-LCL. Apoptosis of EBV-LCL and autologous normal B cells were determined after cultured with different amount of Vδ2-T-Exos for 18 h. All the data shown as mean±SEM are representative of four independent experiments. *p<0.05, **p<0.01. NS, not significant.

FIG. 3B. Vδ2-T-Exos induce apoptosis of EBV-LCL. Active caspase-3 was measured in EBV-LCL after cultured with Vδ2-T-Exos or PBS for 4 h. All the data shown as mean±SEM are representative of four independent experiments. *p<0.05, **p<0.01. NS, not significant.

FIG. 3C. Vδ2-T-Exos induce apoptosis of EBV-LCL. Surface expression of Fas and DR5 on EBV-LCL and autologous normal B cells were determined by flow cytometry. Representative images were shown, and the gray histograms represent isotype controls. All the data shown as mean±SEM are representative of four independent experiments. *p<0.05, **p<0.01. NS, not significant.

FIG. 3D. Vδ2-T-Exos induce apoptosis of EBV-LCL. Vδ2-T-Exos with or without pretreatment with neutralizing anti-FasL, anti-TRAIL antibodies or corresponding isotype control were cultured with EBV-LCL. The apoptosis was calculated as the percentage of inhibition relative to those treated with non-pretreated Vδ2-T-Exos. All the data shown as mean±SEM are representative of four independent experiments. *p<0.05, **p<0.01. NS, not significant.

FIG. 4A. Vδ2-T-Exos control EBV-induced B-cell lymphomas in Rag2^{-/-} γc^{-/-} mice. EBV-induced B-cell lymphoma models were established by injection s.c. of EGFP-expressing EBV-LCL in Rag2^{-/-}γc^{-/-} mice. Vδ2-T-Exos were injected i.p. into Rag2^{-/-}γc^{-/-} mice at indicated time. Equivalent volume of PBS was used as control. Data are expressed as mean±SEM. *p<0.05, **p<0.01. NS, not significant.

FIG. 4B. Vδ2-T-Exos control EBV-induced B-cell lymphomas in Rag2^{-/-} γc^{-/-} mice. Whole-body fluorescence images of mice were determined at 30 days after inoculation s.c. with EBV-LCL (n=6) or PBS (n=6) using an in vivo imaging system. Data are expressed as mean±SEM. *p<0.05, **p<0.01. NS, not significant.

FIG. 4C. Vδ2-T-Exos control EBV-induced B-cell lymphomas in Rag2^{-/-} γc^{-/-} mice. The tumor incidence was measured at the indicated time after treatment with Vδ2-T-Exos or PBS. Data are expressed as mean±SEM. *p<0.05, **p<0.01. NS, not significant.

FIG. 4D. Vδ2-T-Exos control EBV-induced B-cell lymphomas in Rag2^{-/-} γc^{-/-} mice. Representative histology, in situ hybridization of EBER-1/2 and histochemical analysis of human Ki-67 in tumor sections from mice receiving Vδ2-T-Exos or PBS (scale bar=100 nm). Data are expressed as mean±SEM. *p<0.05, **p<0.01. NS, not significant.

FIG. 4E. Vδ2-T-Exos control EBV-induced B-cell lymphomas in Rag2^{-/-} γc^{-/-} mice. The tumor volume was measured at the endpoint. Data are expressed as mean±SEM. *p<0.05, **p<0.01. NS, not significant.

FIG. 4F. Vδ2-T-Exos control EBV-induced B-cell lymphomas in Rag2^{-/-} γc⁴″ mice. The mice survival was determined at indicated time (six mice per group). Data are expressed as mean±SEM. *p<0.05, **p<0.01. NS, not significant.

FIG. 4G. Vδ2-T-Exos control EBV-induced B-cell lymphomas in Rag2^{-/-} γc^{-/-} mice. EGFP-expressing EBV-LCL were injected s.c. in Rag2^{-/-}γc^{-/-} mice. After fourteen days, mice that had developed subcutaneous tumor determined by an in vivo imaging system were randomly divided into two groups followed by the treatment with Vδ2-T-Exos or PBS at indicated time (eight mice per group). Data are expressed as mean±SEM. *p<0.05, **p<0.01. NS, not significant.

FIG. 4H. Vδ2-T-Exos control EBV-induced B-cell lymphomas in Rag2^{-/-} γc^{-/-} mice. Whole-body fluorescence images of mice before treatment with Vδ2-T-Exos or PBS. Data are expressed as mean±SEM. *p<0.05, **p<0.01. NS, not significant.

FIG. 4I. Vδ2-T-Exos control EBV-induced B-cell lymphomas in Rag2^{-/-} γc^{-/-} mice. The mice survival was determined at the indicated time. Data are expressed as mean±SEM. *p<0.05, **p<0.01. NS, not significant.

FIG. 4J. Vδ2-T-Exos control EBV-induced B-cell lymphomas in Rag2^{-/-} γc^{-/-} mice. The tumor volume was determined at the indicated time. Data are expressed as mean±SEM. *p<0.05, **p<0.01. NS, not significant.

FIG. 4K. Vδ2-T-Exos control EBV-induced B-cell lymphomas in Rag2^{-/-} γc^{-/-} mice. Representative histology, in situ hybridization of EBER-1/2 and histochemical analysis of human Ki-67 in tumor sections from mice receiving Vδ2-T-Exos or PBS (scale bar=100 μm). Data are expressed as mean±SEM. *p<0.05, **p<0.01. NS, not significant.

FIG. 5A. Vδ2-T-Exos control EBV-induced B-cell lymphomas in humanized mice. EBV-induced B-cell lymphoma models were established by injection s.c. of autologous EGFP-expressing EBV-LCL in humanized mice. Vδ2-T-Exos allogeneic to the reconstituted huPBMCs were injected into humanized mice i.p. at indicated time. Equivalent volume of PBS was used as control (eight mice per group). Data are expressed as mean±SEM. *p<0.05, **p<0.01. NS, not significant.

FIG. 5B. Vδ2-T-Exos control EBV-induced B-cell lymphomas in humanized mice. The tumor incidence was measured at the endpoint or indicated time. Data are expressed as mean±SEM. *p<0.05, **p<0.01. NS, not significant.

FIG. 5C. Vδ2-T-Exos control EBV-induced B-cell lymphomas in humanized mice. The tumor volume was measured at the endpoint or indicated time. Data are expressed as mean±SEM. *p<0.05, **p<0.01. NS, not significant.

FIG. 5D. Vδ2-T-Exos control EBV-induced B-cell lymphomas in humanized mice. The mice survival was measured at the endpoint or indicated time. Data are expressed as mean±SEM. *p<0.05, **p<0.01. NS, not significant.

FIG. 5E. Vδ2-T-Exos control EBV-induced B-cell lymphomas in humanized mice. Representative histology, in situ hybridization of EBER-1/2 and histochemical analysis of human Ki-67 in tumor sections from mice receiving Vδ2-T-Exos or PBS (scale bar=100 nm). Data are expressed as mean±SEM. *p<0.05, **p<0.01. NS, not significant.

FIG. 5F. Vδ2-T-Exos control EBV-induced B-cell lymphomas in humanized mice. EBV-induced B-cell lymphoma models in Rag2^{-/-}γc^{-/-} mice and humanized mice were established by injection of EBV-LCL s.c. and treated with autologous or allogeneic Vδ2-T-Exos in Rag2^{-/-}γc^{-/-} or humanized mice at the indicated time. Equivalent volume of PBS was used as control (eight mice per group). Data are expressed as mean±SEM. *p<0.05, **p<0.01. NS, not significant.

FIG. 5G. Vδ2-T-Exos control EBV-induced B-cell lymphomas in humanized mice. The tumor incidence was measured at the endpoint or indicated time. Data are expressed as mean±SEM. *p<0.05, **p<0.01. NS, not significant.

FIG. 5H Vδ2-T-Exos control EBV-induced B-cell lymphomas in humanized mice. The tumor volume was measured at the endpoint or indicated time. Data are expressed as mean±SEM. *p<0.05, **p<0.01. NS, not significant.

FIG. 5I. Vδ2-T-Exos control EBV-induced B-cell lymphomas in humanized mice. The mice survival was measured at the endpoint or indicated time. Data are expressed as mean±SEM. *p<0.05, **p<0.01. NS, not significant.

FIG. 5J. Vδ2-T-Exos control EBV-induced B-cell lymphomas in humanized mice. Representative histology, in situ hybridization of EBER-1/2 and histochemical analysis of human Ki-67 in tumor sections from humanized mice receiving autologous Vδ2-T-Exos, allogeneic Vδ2-T-Exos or PBS (scale bar=100 nm). Data are expressed as mean±SEM. *p<0.05, **p<0.01. NS, not significant.

FIG. 5K. Vδ2-T-Exos control EBV-induced B-cell lymphomas in humanized mice. Representative immunofluorescence analysis of human CD3 T cells in tumor sections from humanized mice receiving autologous Vδ2-T-Exos, allogeneic Vδ2-T-Exos or PBS (CD3=red, DAPI=blue; scale bar=20 nm). Data are expressed as mean±SEM. *p<0.05, **p<0.01. NS, not significant.

FIG. 6A. Vδ2-T-Exos induce CD4 and CD8 T cell-mediated antitumor responses. Vδ2-T-Exos were labeled with CFSE and then cultured with CD3 T cells. After 18 h, CFSE signal on CD4 or CD8 T cells was determined by flow cytometry. All the data shown as mean±SEM are representative of four independent experiments. *p<0.05, **p<0.01, ***p<0.001.

FIG. 6B. Vδ2-T-Exos induce CD4 and CD8 T cell-mediated antitumor responses. Vδ2-T-Exos were labeled with CFSE and then cultured with CD3 T cells. After 18 h, CFSE signal on CD4 or CD8 T cells was determined by flow cytometry. All the data shown as mean±SEM are representative of four independent experiments. *p<0.05, **p<0.01, ***p<0.001.

FIG. 6C. Vδ2-T-Exos induce CD4 and CD8 T cell-mediated antitumor responses. Expression of CCR5 on CD4 and CD8 T cells after culture of CD3 T cells with allogeneic Vδ2-T-Exos or PBS for 48 h. All the data shown as mean±SEM are representative of four independent experiments. *p<0.05, **p<0.01, ***p<0.001.

FIG. 6D. Vδ2-T-Exos induce CD4 and CD8 T cell-mediated antitumor responses. Expression of CCR5 on CD4 and CD8 T cells after culture of CD3 T cells with allogeneic Vδ2-T-Exos or PBS for 48 h. All the data shown as mean±SEM are representative of four independent experiments. *p<0.05, **p<0.01, ***p<0.001.

FIG. 6E. Vδ2-T-Exos induce CD4 and CD8 T cell-mediated antitumor responses. The Vδ2-T-Exos-pretreated CD3 T cells were incubated with neutralizing anti-CCR5 antibody or isotype control for 30 min and added in the upper chamber. PBS-pretreated CD3 T cells were used as control. The supernatants from EBV-LCL were added in the bottom chamber. The relative percentages of cells migrated from the upper chamber after 4 h are shown. All the data shown as mean±SEM are representative of four independent experiments. *p<0.05, **p<0.01, ***p<0.001.

FIG. 6F. Vδ2-T-Exos induce CD4 and CD8 T cell-mediated antitumor responses. Proliferation and intracellular expression of IFN-γ in CD4 or CD8 T cells after 7 days culture of CD3 T cells with different amount of autologous or allogeneic Vδ2-T-Exos. Left, representative images of flow cytometry. All the data shown as mean±SEM are representative of four independent experiments. *p<0.05, **p<0.01, ***p<0.001.

FIG. 6G. Vδ2-T-Exos induce CD4 and CD8 T cell-mediated antitumor responses. Proliferation and intracellular expression of IFN-γ in CD4 or CD8 T cells after 7 days culture of CD3 T cells with different amount of autologous or allogeneic Vδ2-T-Exos. Left, representative images of flow cytometry. All the data shown as mean±SEM are representative of four independent experiments. *p<0.05, **p<0.01, ***p<0.001.

FIG. 6H. Vδ2-T-Exos induce CD4 and CD8 T cell-mediated antitumor responses. Proliferation and intracellular expression of IFN-γ in CD4 or CD8 T cells after 7 days culture of CD3 T cells with different amount of autologous or allogeneic Vδ2-T-Exos. Left, representative images of flow cytometry. All the data shown as mean±SEM are representative of four independent experiments. *p<0.05, **p<0.01, ***p<0.001.

FIG. 6I. Vδ2-T-Exos induce CD4 and CD8 T cell-mediated antitumor responses. Proliferation and intracellular expression of IFN-γ in CD4 or CD8 T cells after 7 days culture of CD3 T cells with different amount of autologous or allogeneic Vδ2-T-Exos. Left, representative images of flow cytometry. All the data shown as mean±SEM are representative of four independent experiments. *p<0.05, **p<0.01, ***p<0.001.

FIG. 6J. Vδ2-T-Exos induce CD4 and CD8 T cell-mediated antitumor responses. The EBV-specific cytotoxic T lymphocytes (EBV-CTLs) were selected from EBV-seropositive huPBMCs and cultured with allogeneic Vδ2-T-Exos or PBS in the presence of IL-2. Two weeks later, the cell number of EBV-CTLs were determined by the intracellular staining of IFN-γ and counted by counting beads. All the data shown as mean±SEM are representative of four independent experiments. *p<0.05, **p<0.01, ***p<0.001. The EBV-specific cytotoxic T lymphocytes (EBV-CTLs) were selected from EBV-seropositive huPBMCs and cultured with allogeneic Vδ2-T-Exos or PBS in the presence of IL-2. Two weeks later, the cell number of EBV-CTLs were determined by the intracellular staining of IFN-γ and counted by counting beads. All the data shown as mean±SEM are representative of four independent experiments. *p<0.05, **p<0.01, ***p<0.001.

FIG. 6K. Vδ2-T-Exos induce CD4 and CD8 T cell-mediated antitumor responses. The EBV-specific cytotoxic T lymphocytes (EBV-CTLs) were selected from EBV-seropositive huPBMCs and cultured with allogeneic Vδ2-T-Exos or PBS in the presence of IL-2. Two weeks later, the cell number of EBV-CTLs were determined by the intracellular staining of IFN-γ and counted by counting beads. All the data shown as mean±SEM are representative of four independent experiments. *p<0.05, **p<0.01, ***p<0.001. The EBV-specific cytotoxic T lymphocytes (EBV-CTLs) were selected from EBV-seropositive huPBMCs and cultured with allogeneic Vδ2-T-Exos or PBS in the presence of IL-2. Two weeks later, the cell number of EBV-CTLs were determined by the intracellular staining of IFN-γ and counted by counting beads. All the data shown as mean±SEM are representative of four independent experiments. *p<0.05, **p<0.01, ***p<0.001.

FIG. 7A. CD4 and CD8 T cells are involved in Vδ2-T-Exos-induced antitumor immunity in humanized mice. EBV-induced B-cell lymphoma models were established by injection of autologous EBV-LCL in humanized mice reconstituted with whole huPBMCs, CD4-T-cell-depleted huPBMCs, or CD8-T-cell-depleted huPBMCs from same donors. Allogeneic Vδ2-T-Exos were injected into humanized mice i.p. at indicated time (eight mice per group). Data are expressed as mean±SEM. *p<0.05, NS, not significant.

FIG. 7B. CD4 and CD8 T cells are involved in Vδ2-T-Exos-induced antitumor immunity in humanized mice. The tumor incidence was measured at the endpoint or indicated time. Data are expressed as mean±SEM. *p<0.05, NS, not significant.

FIG. 7C. CD4 and CD8 T cells are involved in Vδ2-T-Exos-induced antitumor immunity in humanized mice. The tumor volume was measured at the endpoint or indicated time. Data are expressed as mean±SEM. *p<0.05, NS, not significant.

FIG. 7D. CD4 and CD8 T cells are involved in Vδ2-T-Exos-induced antitumor immunity in humanized mice. The mice survival was measured at the endpoint or indicated time. Data are expressed as mean±SEM. *p<0.05, NS, not significant.

FIG. 8A. Characteristics of Vδ2-T-Exos. Iodixanol gradient separation of extracellular vesicles derived from Vδ2-T cells into 12 sub-fractions. Representative data are shown as mean±SEM four independent experiments. **p<0.01.

FIG. 8B. Characteristics of Vδ2-T-Exos. Western blot analysis of exosomal makers CD81, TSG101, CD63 and Alix in the sub-fractions after iodixanol gradient floatation. Representative data are shown as mean±SEM four independent experiments. **p<0.01.

FIG. 8C. Characteristics of Vδ2-T-Exos. Apoptosis of EBV-LCL after cultured with the gradient sub-fractions for 18 h. Representative data are shown as mean±SEM four independent experiments. **p<0.01.

FIG. 9. Activation and functional markers on Vδ2-T cells. Cell surface markers as indicated were determined by flow cytometry on resting Vδ2-T cells (day 0) or PAM-expanded Vδ2-T cells (day 16). The gray histograms represent isotype control. Data are shown as representative of four independent experiments.

FIG. 10A. Roles of HLA and CD86 in the Vδ2-T-Exos induced T cells responses. Proliferation in CD4 T cells after culture of CD3 T cells with allogeneic Vδ2-T-Exos, neutralizing anti-HLA-DR/DP/DQ antibody or isotype control pretreated Vδ2-T-Exos. All the data shown as mean±SEM are representative of four independent experiments. *p<0.05, **p<0.01. NS, not significant.

FIG. 10B. Roles of HLA and CD86 in the Vδ2-T-Exos induced T cells responses. Intracellular expression of IFN-γ in CD4 T cells after culture of CD3 T cells with allogeneic Vδ2-T-Exos, neutralizing anti-HLA-DR/DP/DQ antibody or isotype control pretreated Vδ2-T-Exos. All the data shown as mean±SEM are representative of four independent experiments. *p<0.05, **p<0.01. NS, not significant.

FIG. 10C. Roles of HLA and CD86 in the Vδ2-T-Exos induced T cells responses. Proliferation in CD8 T cells after culture of CD3 T cells with allogeneic Vδ2-T-Exos, neutralizing anti-HLA-A/B/C antibody or isotype control pretreated Vδ2-T-Exos. All the data shown as mean±SEM are representative of four independent experiments. *p<0.05, **p<0.01. NS, not significant.

FIG. 10D. Roles of HLA and CD86 in the Vδ2-T-Exos induced T cells responses. Intracellular expression of IFN-γ in CD8 T cells after culture of CD3 T cells with allogeneic Vδ2-T-Exos, neutralizing anti-HLA-A/B/C antibody or isotype control pretreated Vδ2-T-Exos. All the data shown as mean±SEM are representative of four independent experiments. *p<0.05, **p<0.01. NS, not significant.

FIG. 10E. Roles of HLA and CD86 in the Vδ2-T-Exos induced T cells responses. Proliferation in CD4 T cells after culture of CD3 T cells with allogeneic Vδ2-T-Exos, neutralizing anti-CD86 antibody or isotype control pretreated Vδ2-T-Exos. All the data shown as mean±SEM are representative of four independent experiments. *p<0.05, **p<0.01. NS, not significant.

FIG. 10F. Roles of HLA and CD86 in the V 62-T-Exos induced T cells responses. Intracellular expression of IFN-γ in CD4 T cells after culture of CD3 T cells with allogeneic Vδ2-T-Exos, neutralizing anti-CD86 antibody or isotype control pretreated Vδ2-T-Exos. All the data shown as mean±SEM are representative of four independent experiments. *p<0.05, **p<0.01. NS, not significant.

FIG. 10G. Roles of HLA and CD86 in the Vδ2-T-Exos induced T cells responses. Proliferation in CD8 T cells after culture of CD3 T cells with allogeneic Vδ2-T-Exos, neutralizing anti-CD86 antibody or isotype control pretreated Vδ2-T-Exos. All the data shown as mean±SEM are representative of four independent experiments. *p<0.05, **p<0.01. NS, not significant.

FIG. 10H. Roles of HLA and CD86 in the Vδ2-T-Exos induced T cells responses. Intracellular expression of IFN-γ in CD8 T cells after culture of CD3 T cells with allogeneic Vδ2-T-Exos, neutralizing anti-CD86 antibody or isotype control pretreated Vδ2-T-Exos. All the data shown as mean±SEM are representative of four independent experiments. *p<0.05, **p<0.01. NS, not significant.

FIG. 11. Surface expression of MICA/B on EBV-LCL and autologous normal B cells. The expression of MICA/B on EBV-LCL and autologous normal B cells was determined by flow cytometry, the gray histograms represent isotype controls. Data are representative for four independent experiments.

FIGS. 12A-12E. Vδ2-T-Exos isolated according to the methods disclosed in Example 8.

### DETAILED DISCLOSURE OF THE INVENTION

As used herein, the singular forms “a,” “an,” and “the” are intended to include the plural forms as well, unless the context clearly indicates otherwise. Furthermore, to the extent that the terms “including,” “includes,” “having,” “has,” “with,” or variants thereof are used in either the detailed description and/or the claims, such terms are intended to be inclusive in a manner similar to the term “comprising.” The transitional terms/phrases (and any grammatical variations thereof) “comprising,” “comprises,” “comprise,” “consisting essentially of,” “consists essentially of,” “consisting,” and “consists” can be used interchangeably.

The phrases “consisting essentially of” or “consists essentially of” indicate that the claim encompasses embodiments containing the specified materials or steps and those that do not materially affect the basic and novel characteristic(s) of the claim.

The term “about” means within an acceptable error range for the particular value as determined by one of ordinary skill in the art, which will depend in part on how the value is measured or determined, i.e., the limitations of the measurement system. Typically, “about” can mean a range of up to 0-10% of a given value.

In the present disclosure, ranges are stated in shorthand to avoid having to set out at length and describe each and every value within the range. Any appropriate value within the range can be selected, where appropriate, as the upper value, lower value, or the terminus of the range. For example, a range of 0.1-1.0 represents the terminal values of 0.1 and 1.0, as well as the intermediate values of 0.2, 0.3, 0.4, 0.5, 0.6, 0.7, 0.8, 0.9, and all intermediate ranges encompassed within 0.1-1.0, such as 0.2-0.5, 0.2-0.8, 0.7-1.0, etc. Values having at least two significant digits within a range are envisioned, for example, a range of 5-10 indicates all the values between 5.0 and 10.0 as well as between 5.00 and 10.00 including the terminal values.

“Treatment,” or “treating” (and grammatical variants of these terms), as used herein, are used interchangeably. These terms refer to an approach for obtaining beneficial or desired results including but not limited to therapeutic benefit. A therapeutic benefit is achieved with the eradication or amelioration of one or more of the physiological symptoms associated with the underlying cancer such that an improvement is observed in the patient, notwithstanding that the patient may still be afflicted with the underlying cancer.

The term “effective amount” or “therapeutically effective amount” of the exosomes refers to that amount of the exosomes described herein that is sufficient to effect the intended application including but not limited to cancer treatment. The therapeutically effective amount may vary depending upon the subject and disease condition being treated, e.g., the weight and age of the subject, the severity of the cancer, the manner of administration and the like, which can readily be determined by one of ordinary skill in the art. The term also applies to a dose that will induce a particular response in target cells, e.g., killing or reduction of proliferation of the target cells. The specific dose will also vary depending on the dosing regimen to be followed, whether it is administered in combination with other compounds, timing of administration, the tissue to which it is administered, and the physical delivery system in which it is carried.

“Subject” refers to an animal, such as a mammal, for example a human. The methods described herein can be useful in both pre-clinical human therapeutics and veterinary applications. In some embodiments, the subject is a mammal (such as an animal model of disease), and in some embodiments, the subject is human. The terms “subject” and “patient” can be used interchangeably.

Exosomes are endosome-originated small extracellular vesicles (20-200 nm) that shuttle lipid, proteins, and nucleic acid in intercellular communication. They have high bioavailability, biostability, biocompatibility, and cargo loading capacity. Exosomes can be engineered to achieve targeting specificity, which makes them powerful nanocarriers to deliver antitumor agents and induce antigen-specific antitumor immunity.

Vδ2⁺ T cells belong to a subset of T lymphocytes. Vδ2⁺ T cells exist in the peripheral blood and lymphoid organs, and generally co-express Vγ9. Vδ2⁺ T cells can be activated and expanded in a MHC-independent manner by phosphoantigens, the small nonpeptidic phosphorylated intermediates of mevalonate pathway in mammalian cells. PAM, a pharmacological aminobisphosphonate commonly used for the treatment of osteoporosis, can also selectively activate and expand human Vδ2⁺ T cells in vitro and in vivo.

Typically, an EBV infection is asymptomatic because a host's immune system controls the infection; however, some individuals may develop self-limiting infectious mononucleosis, while others may develop EBV-associated lymphoid or epithelial cancers. The EBV life cycle includes a lytic phase that results in the production of new viral particles, and a latent phase when the virus remains largely silent for the lifetime of the host in memory B cells. Therefore, an EBV-infected cell can have an EBV-virus in the lytic phase or in the latent phase.

For the purpose of this invention, the phrase “exosomes from Vδ2⁺ T cells” refers to exosomes isolated from Vδ2⁺ T cells. These exosomes can be isolated from Vδ2⁺ T cells obtained from a subject to be treated for an EBV-induced cancer, such as an EBV-induced B-cell cancer. These exosomes can also be isolated from a healthy individual. The exosomes can be isolated from Vδ2⁺ T cells obtained from a subject before or after activating the Vδ2⁺ T cells in vitro. Typically, Vδ2⁺ T cells are activated in vitro in the presence of a phosphoantigen, such as isopentenyl pyrophosphate (IPP), (E)-4-hydroxy-3-methyl-but-2-enyl-pyrophosphate (HMB-PP), bromohydrin pyrophosphate (BrHPP), and PAM. Additional examples of phosphoantigens that can activate Vδ2⁺ T cells are known in the art and such embodiments are within the purview of the invention.

Certain methods for isolating exosomes are known in the art and can be used to isolate exosomes from the Vδ2⁺ T cells. Typically, supernatant from cultured Vδ2⁺ T cells are used as a source of exosomes.

Dendritic cells (DC) derived exosomes (DC-Exos) or natural killer cells derived exosomes (NK-Exos)-based immunotherapies have shown promise for cancer therapy, but their antitumor efficacy was limited in some cancer patients. The heterogeneity of ex vivo expanded DC may be partially account for the poor therapeutic outcome of DC-Exos-based therapy. Exosomes derived from immature DCs may have immune tolerizing activities. The conventional approaches are difficult to generate highly homogeneous human DC, and their maturation is usually incomplete and asynchronous. Hence, tolerogenic DC-Exos are often co-isolated with immunostimulatory DC-Exos and worsen their therapeutic outcomes. In addition, it still remains a challenge to expand DC or NK cells ex vivo in large scale. In contrast, optimized protocols are known for ex vivo expansion of homogeneous human Vδ2-T cells in clinical scale by using phosphoantigens, which allows for producing large amounts of Vδ2-T-Exos. Most importantly, the cytolytic activities against tumor cells and immunostimulatory properties of Vδ2-T cells can be maintained even after long-term expansion.

While certain other exosomes, such as DC-Exos or NK-Exos are tested for their ability to target and kill cancer cells, the antitumor activity of Vδ2-T cell-derived exosomes (Vδ2-T-Exos) remains unknown. This disclosure provides that Vδ2-T-Exos preserved the antitumor activities of Vδ2-T cells while avoiding the limitations of cell-based cancer immunotherapy. Particularly, human Vδ2-T-Exos efficiently induced EBV-LCL apoptosis in vitro, and inhibited the development of EBV-induced B-cell lymphomas in Rag2^{-/-}γc^{-/-} and humanized mice. Allogeneic Vδ2-T-Exos had more potent antitumor activity than autologous Vδ2-T-Exos, probably, because they could induce more robust CD4 and CD8 T cells-medicated antitumor immunity.

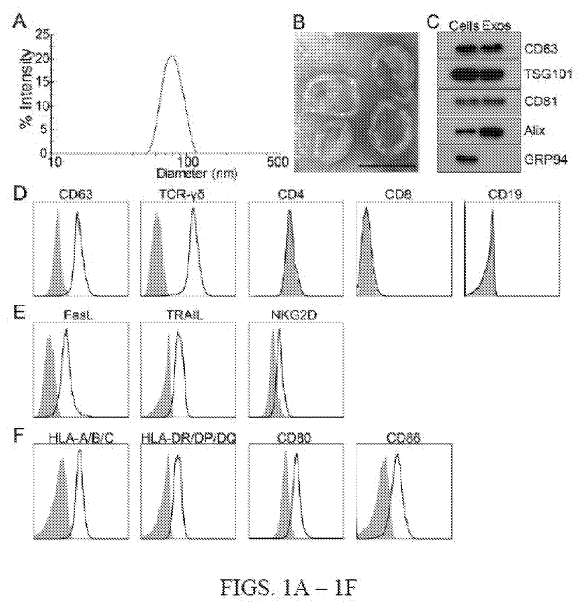

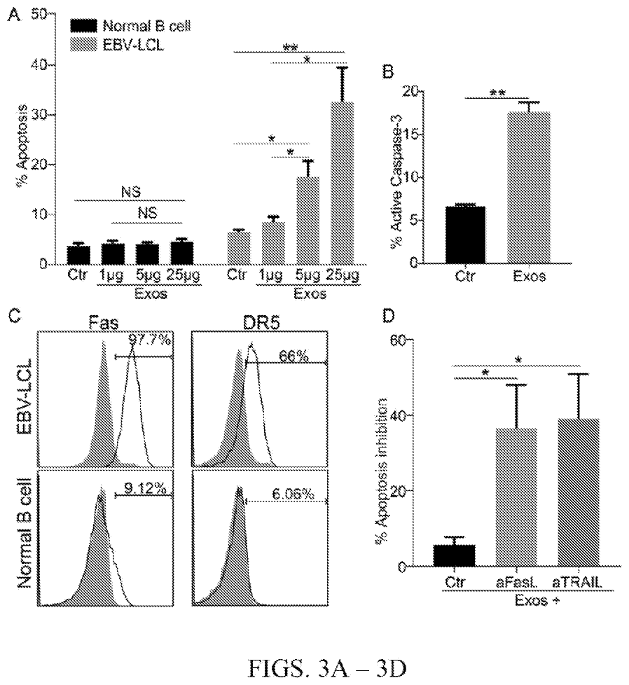

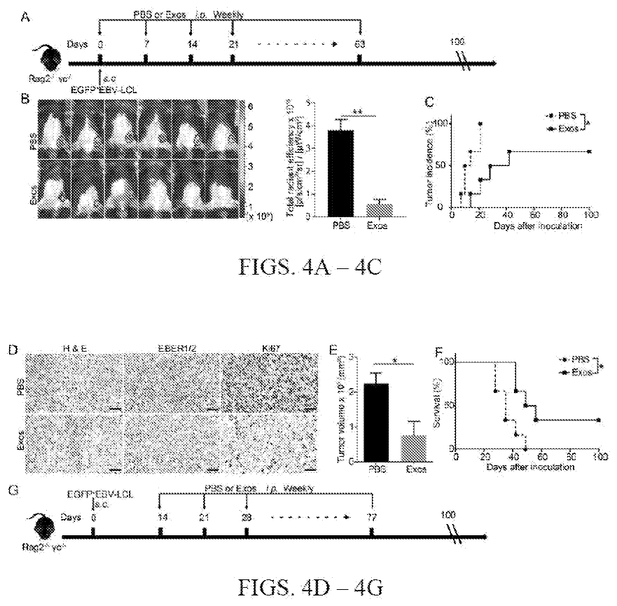

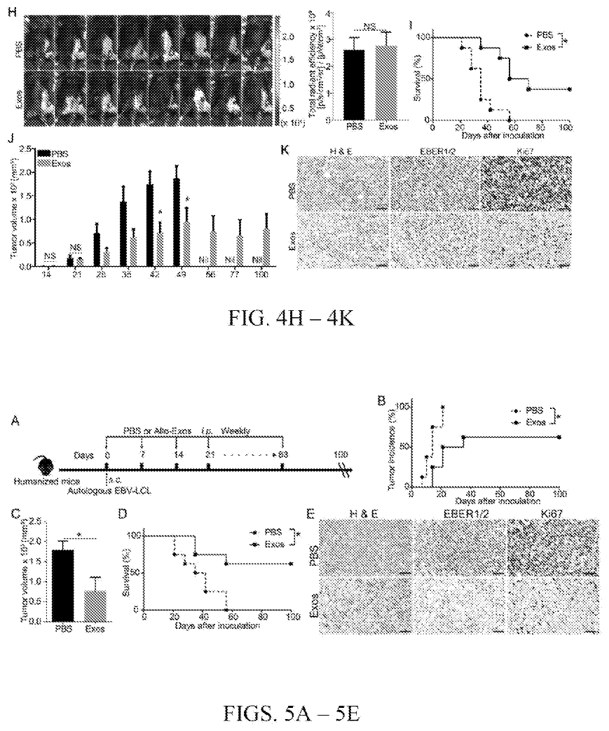

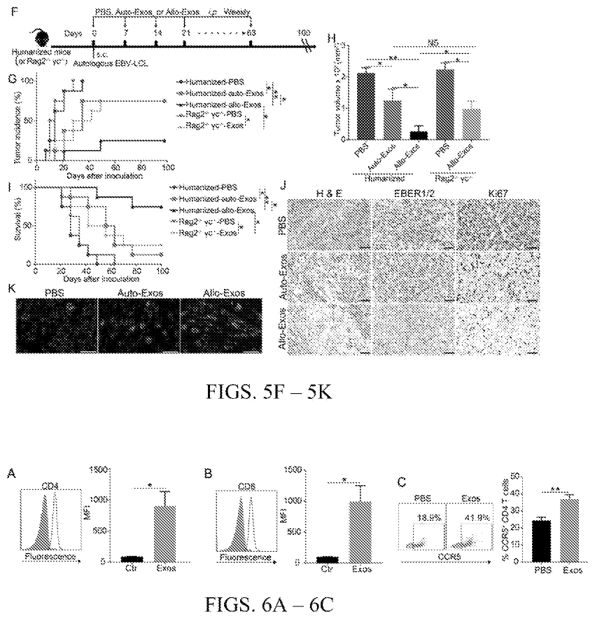

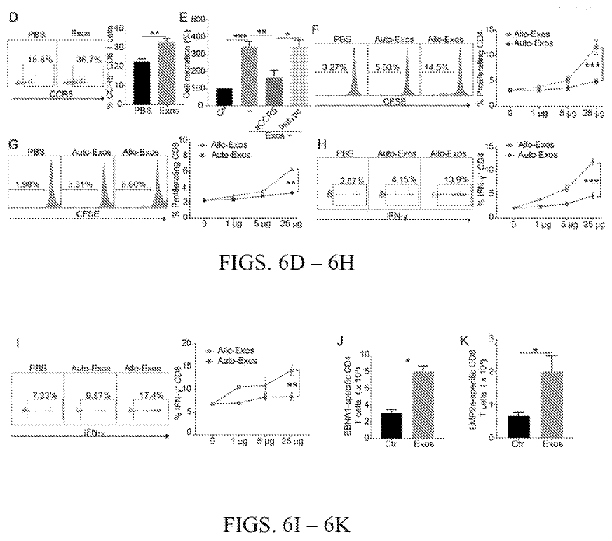

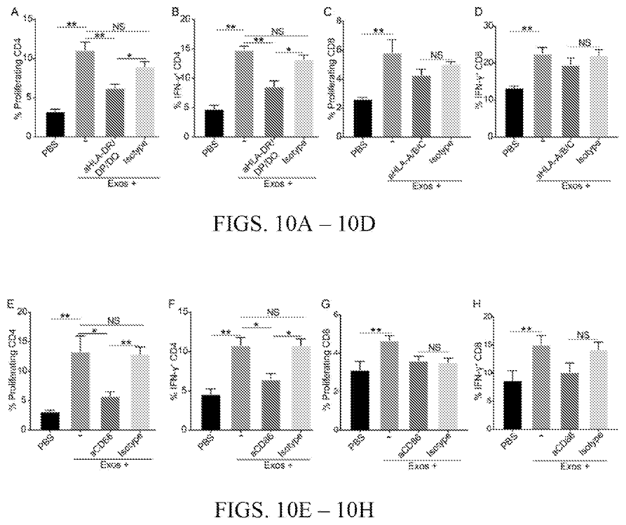

Similar to exosomes derived from other cells, the surface of Vδ2-T-Exos was decorated with intact functional molecules from their parent cells. Since human Vδ2-T cells share the characteristics of NK and DC, Vδ2-T-Exos may have dual antitumor activities. Like exosomes derived from NK cells, Vδ2-T-Exos were found to carry FasL, and TRAIL (FIG. 1), which could interact with Fas and DR5 expressed on EBV-LCL (FIG. 3) respectively, and then induced EBV-LCL apoptosis (FIG. 3), thus efficiently inhibited the development and progression of EBV-induced B-cell lymphomas in Rag2^{-/-}γc^{-/-} (FIG. 4) and humanized mice (FIG. 5) through Fas/FasL and TRAIL/DR5 pathways. Similar to exosomes derived from DC, Vδ2-T-Exos also retained the essential immunostimulatory and MHC-I/II molecules that are required for antigen presentation and T cell priming, such as CD80, CD86, HLA-A/B/C and HLA/DR/DP/DQ (FIG. 1). Indeed, Vδ2-T-Exos could enhance cell proliferation and IFN-γ production in CD4 cells through the recognition of HLA-DR/DP/DQ and CD86 (FIG. 6, FIG. 10). Furthermore, both CD4 and CD8 T cells were involved in the Vδ2-T-Exos-mediated antitumor immunity against EBV-induced B-cell lymphoma in humanized mice (FIG. 7). Therefore, Vδ2-T-Exos have multiple antitumor activities and share the antitumor properties of NK-Exos and DCs-Exos.

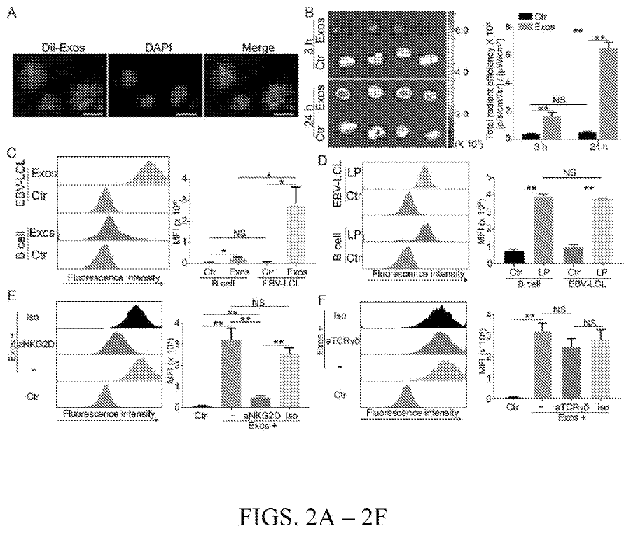

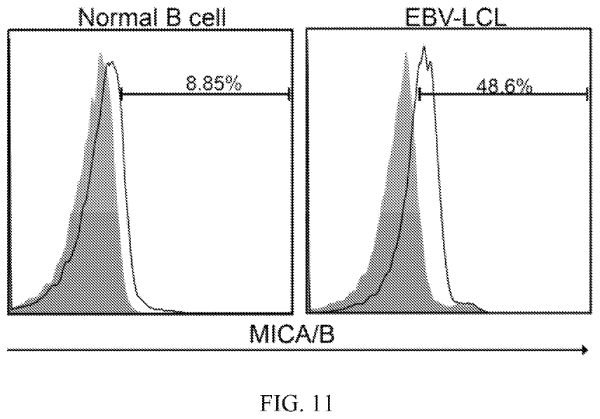

This disclosure shows that Vδ2-T-Exos can target EBV-LCL through the interaction of Vδ2-T-Exos carried NKG2D and its ligands MICA/B which constitutively expressed on EBV-LCL (FIG. 2E and FIG. 11). This targeting was not dependent on Vδ2-T-Exos carried TCR-γδ (FIG. 2F). As a low pH condition could improve the uptake of exosomes by tumor cells, the acidic condition in tumor microenvironment, a hallmark of tumor malignancy, may also account for the accumulation of Vδ2-T-Exos in tumor site.

Importantly, allogeneic Vδ2-T-Exos could increase the infiltration of T cells in EBV-induced tumor tissues through the upregulation of CCR5 on T cells because EBV-induced lymphoma cells could secrete abundant CCR5 ligands. In addition, allogeneic Vδ2-T-Exos were more potent to induce cell proliferation and IFN-γ production in CD4 and CD8 T cells than autologous Vδ2-T-Exos (FIG. 6H-K), thus allogeneic Vδ2-T-Exos had better therapeutic effect than autologous Vδ2-T-Exos on EBV-induced B-cell lymphomas in humanized mice (FIG. 5F-J). Indeed, once blockade of HLA-DR/DP/DQ molecules carried on allogeneic Vδ2-T-Exos, CD4 T cell responses induced by Vδ2-T-Exos could be significantly abrogated (FIG. 10), indicating that allo-recognition of HLA-DR/DP/DQ molecules on allogeneic Vδ2-T-Exos plays an important role in the induction of T cell response.

Thus, the disclosure provides a novel therapeutic strategy using Vδ2-T-Exos to treat EBV-induced B-cell lymphomas. As a cell-free therapy, Vδ2-T-Exos take the advantages of both NK-Exos and DCs-Exos by inheriting the cytotoxic and immunostimulatory properties from Vδ2-T cells, which allow them to effectively control EBV-induced B-cell lymphomas. Vδ2-T-Exos-based therapy, especially allogeneic Vδ2-T-Exos-based therapy, has great potential to overcome the shortcoming of conventional immunotherapies for EBV-induced B-cell lymphomas.

Accordingly, certain embodiments of the invention provide a method for killing or inhibiting the growth of an EBV-infected cell, comprising contacting the EBV-infected cell with exosomes from Vδ2⁺ T cells in an amount effective to kill or inhibit the growth of the cell. In certain embodiments, the EBV-infected cell is an EBV-infected lymphocyte, such as a B-lymphocyte. In other embodiments, the EBV-infected cell is an EBV-infected epithelial cell. In preferred embodiments, the EBV-infected cell has become neoplastic, such as an EBV-infected neoplastic B-lymphocyte or an EBV-infected neoplastic epithelial cell.

In certain embodiments, the exosomes are isolated from Vδ2⁺ T cells that are autologous to the EBV-infected cell. Preferably, the exosomes are isolated from Vδ2⁺ T cells that are allogeneic to the EBV-infected cell.

Further embodiments of the invention provide a method of treating an EBV-induced cancer, comprising administering to a subject in need thereof a therapeutically effective amount of exosomes from Vδ2⁺ T cells.

For the purposes of this invention, the phrase “EBV-induced cancer” refers to a cancer that results from an EBV-infected cell that has become neoplastic. EBV typically infects lymphocytes or epithelial cells. Therefore, the disclosure provides methods of treating a cancer of lymphocytic origin or epithelial origin.

Lymphocytes commonly infected by EBV are B-cells. Therefore, the disclosure provides a method of treating an EBV-induced neoplasm, such as EBV-induced B-cell neoplasm including EBV-induced: Burkitt lymphoma, Hodgkin's lymphoma, diffuse large B-cell lymphoma, and lymphoproliferative disease.

The disclosure also provides a method of treating an EBV-induced epithelial cancer, such as EBV-induced nasopharyngeal carcinoma (NPC) or EBV-induced gastric cancer/carcinoma.

As most cancer patients are immunocompromised, it is difficult to expand their Vδ2-T cells and prepare autologous Vδ2-T-Exos ex vivo in large scale. In addition, the compositions of Vδ2-T-Exos from different patients are also varied which may cause the variation of their therapeutic effects. In contrast, it is convenient to expand and prepare allogeneic Vδ2-T-Exos ex vivo in large scale from healthy individuals by currently available protocols. Allogeneic Vδ2-T cells could control tumor growth without side effects in cholangiocarcinoma patients. As the phosphoantigens-expanded Vδ2-T cells display a homogeneous antitumor property, pooling allogeneic Vδ2-T-Exos together from a large number of healthy individuals may be beneficial to quality control, standardization and centralization. Therefore, cancer therapy based on allogeneic rather than autologous Vδ2-T-Exos may be more efficient and feasible in future clinical practice.

About 1-10% of T lymphocytes of an individual can respond to the foreign MHC molecules through direct T-cell allo-recognition. These high frequency precursors also have specificity for the antigens presented by self MHC molecules, which have been found on several occasions for viral peptides and encompasses both CD4 and CD8 T cells. Further, the allogeneic response may promote an effective T-cell response to self HLA-restricted tumor antigen and reverse the exhaustion of pre-existing antigen-specific cytotoxic T lymphocytes, which can consequently boost the immune eradication of tumor cells. Therefore, such cross-reactions can be proposed for the treatment of virus infection and cancers. The disclosure also shows that allogeneic Vδ2-T-Exos could promote the expansion of pre-existing EBV-specific CD4 and CD8 T cells, which could benefit for the antitumor efficacy of Vδ2-T-Exos against EBV-induced B-cell lymphomas.

Accordingly, while exosomes can be isolated from Vδ2-T cells from the subject suffering from a cancer, in preferred embodiments, the invention provides methods of treating an EBV-induced cancer in a subject by administering exosomes that are obtained from Vδ2⁺ T cells from an individual, preferably, an healthy individual, who is allogeneic to the subject.

Alternatively, the exosomes can be obtained from Vδ2⁺ T cells from the subject when the subject was known to be free of cancer. Thus, exosomes can be obtained from Vδ2⁺ T cells from the subject and stored under appropriate conditions, for example, frozen, and administered to the subject if the subject develops an EBV-induced cancer.

The exosomes can be administered to a subject via any convenient and effective route of administration, such oral, rectal, nasal, topical, (including buccal and sublingual), transdermal, vaginal, parenteral (including intramuscular, subcutaneous, and intravenous), spinal (epidural, intrathecal), and central (intracerebroventricular) administration.

Further embodiments of the invention provide a method for isolating Vδ2-T-Exos. In certain embodiments, the method comprises the steps of:

a) providing peripheral mononuclear cells (PBMCs),

b) culturing the PBMCs in a culture medium in the presence of a phosphoantigen and IL-2 for a first period of time,
c) after the first period of time, culturing the PBMCs in an exosome free culture medium in the presence of PAM and IL-2 for a second period of time,

d) isolating the exosomes from the culture supernatant after the second period.

In preferred embodiments, the PBMCs are human PBMCs. Typically, human PMBCs are cultured in the presence of a phosphoantigen and IL-2 for a period of between 14 and 20 days. After this culturing period, the cells are cultured in a fresh medium free from exosomes for an additional period of between 24 hours to 72 hours, preferably, about 48 hours, also in the presence of a phosphoantigen and IL-2.

The exosomes can be isolated at the end of the second culturing period by various steps known in the art to isolate exosomes. Such steps can include filtration, centrifugation, ultracentrifugation, and a combination thereof.

The phosphoantigens that could be used in the methods of isolating Vδ2-T-Exos include isopentenyl pyrophosphate (IPP), (E)-4-hydroxy-3-methyl-but-2-enyl-pyrophosphate (HMB-PP), bromohydrin pyrophosphate (BrHPP), Pamidronate (PAM), or any combination thereof. Additional examples of phosphoantigens that stimulate Vδ2-T cells are known in the art and such embodiments are within the purview of the invention.

Further embodiments of the invention provide a pharmaceutical composition comprising Vδ2-T-Exos isolated according to the method comprising the steps of:

a) providing peripheral mononuclear cells (PBMCs),

b) culturing the PBMCs in a culture medium in the presence of a phosphoantigen and IL-2 for a first period of time,

c) after the first period of time, culturing the PBMCs in an exosome free culture medium in the presence of PAM and IL-2 for a second period of time,

d) isolating the exosomes from the culture supernatant after the second period.

The details provided above regarding the methods of isolating Vδ2-T-Exos are also applicable to the pharmaceutical compositions of the invention.

In addition to the Vδ2-T-Exos isolated according to the methods disclosed herein, the pharmaceutical compositions can further contain a pharmaceutically acceptable carrier. “Pharmaceutically acceptable carrier” refers to a diluent, adjuvant, or excipient with which are formulated the Vδ2-T-Exos isolated according to the methods disclosed herein. Typically, a “pharmaceutically acceptable carrier” is a substance that is non-toxic, biologically tolerable, and otherwise biologically suitable for administration to a subject, such as an inert substance, added to a pharmacological composition or otherwise used as a diluent, adjuvant, or excipient to facilitate administration of the Vδ2-T-Exos isolated according to the methods disclosed herein and that is compatible therewith. Examples of excipients include various sugars and types of starches, cellulose derivatives, gelatin, vegetable oils, and polyethylene glycols. Additional examples of carriers suitable for use in the pharmaceutical compositions are known in the art and such embodiments are within the purview of the invention.

The pharmaceutical compositions of the invention can be formulated for administration to a subject via any convenient and effective route, such oral, rectal, nasal, topical, (including buccal and sublingual), transdermal, vaginal, parenteral (including intramuscular, subcutaneous, and intravenous), spinal (epidural, intrathecal), and central (intracerebroventricular) administration.

Additional embodiments of the invention provide methods for killing or inhibiting the growth of an EBV-infected cell, comprising contacting the EBV-infected cell with the Vδ2-T-Exos isolated according to the Vδ2-T-Exos isolation methods disclosed herein in an amount effective to kill or inhibit the growth of the cell.

Further embodiments of the invention also provide methods of treating an EBV-induced cancer, comprising administering to a subject in need thereof a therapeutically effective amount of the Vδ2-T-Exos isolated according to the Vδ2-T-Exos isolation methods disclosed herein.

Certain aspects of killing or inhibiting the growth of an EBV-infected cell by contacting the cell with Vδ2-T-Exos are discussed above, such as the type of cell EBV-infected cell and the source Vδ2⁺ T cells of Vδ2-T-Exos. These aspects also apply to the methods for killing or inhibiting the growth of an EBV-infected cell, comprising contacting the EBV-infected cell with the Vδ2-T-Exos isolated according to the Vδ2-T-Exos isolation methods disclosed herein.

Similarly, the aspects of treating an EBV-induced cancer, comprising administering to a subject in need thereof a therapeutically effective amount of Vδ2-T-Exos are discussed above, such as the type of cancer, the source Vδ2⁺ T cells of Vδ2-T-Exos, the route of administration, and the subject. These aspects also apply to the methods for treating an EBV-induced cancer, comprising administering to a subject in need thereof a therapeutically effective amount of the Vδ2-T-Exos isolated according to the Vδ2-T-Exos isolation methods disclosed herein.

### Materials and Methods

### Study Design

The aim of this study was to determine the antitumor effects of Vδ2-T-Exos against EBV-induced B-cell lymphomas. We characterized immune molecules with antitumor potentials on Vδ2-T-Exos and determined the interaction of Vδ2-T-Exos with EBV-LCL. We then evaluated the antitumor effects of Vδ2-T-Exos against EBV-induced B-cell lymphomas in vitro, in immunedeficient mice and humanized mice. Considering the feasibility of clinical application, we then compared the antitumor effects of autologous Vδ2-T-Exos with allogeneic Vδ2-T-Exos in humanized mice. The effects of Vδ2-T-Exos on T cell response and underlying mechanism were further investigated. Finally, we compared the antitumor effects of Vδ2-T-Exos in humanized mice reconstituted with whole huPBMC, CD4-depleted huPBMC and CD8-depleted huPBMC. Mice were age- and gender-matched between groups. The sample sizes were determined by investigator upon previous experience. Sample size and replication were specified in figure legends. All the research protocols and animal work were approved by The Institutional Review Board of the University of Hong Kong/Hospital Authority Hong Kong West Cluster and the Committee on the Use of Live Animals in Teaching and Research, The University of Hong Kong.

### Isolation and Characterization of Vδ2-T-Exos

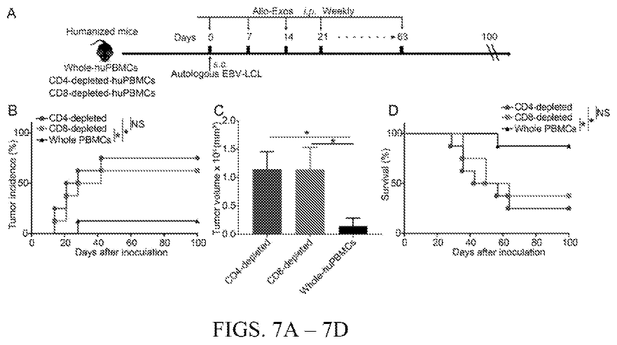

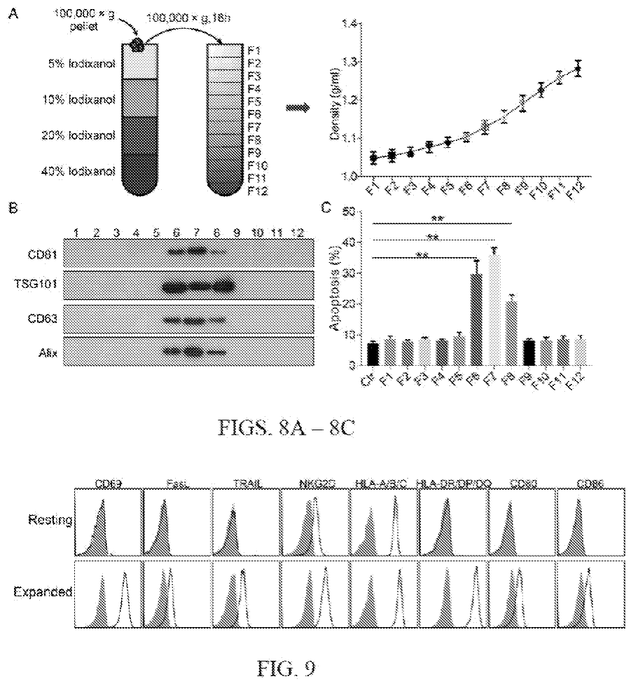

To produce Vδ2-T-Exos, PAM-expanded Vδ2-T cells were cultured in exosome-free medium for 48 hours. The conditioned medium was then collected and subjected to differential ultracentrifugation. Dynamic light scattering showed that the ultracentrifuged pellets displayed a bell-shaped size distribution profile which represented a homogeneous population with a peak at 80 nm (FIG. 1A). Electronic microscopy analysis revealed that the ultracentrifuged pellets contained vesicles that resembled exosomes in cup-shaped morphology (FIG. 1B). Western blot analysis indicated that these small vesicles were positive for exosomal markers CD63, TSG101, CD81, Alix, whereas negative for endoplasmic reticulum protein GRP94 (FIG. 1C). Density gradient ultracentrifugation of these small vesicles further demonstrated that they primarily consisted of exosomes, as evidenced by expression of exosomal markers largely in fraction 6, 7 and 8 (FIGS. 8A and 8B). The expression of TCR-γδ confirmed that these small vesicles originated from Vδ2-T cells, and the absence of CD4, CD8 and CD19 expressions excluded the potential contamination of vesicles from other immune cells (FIG. 1D). Similar as their parental PAM-expanded Vδ2-T cells (FIG. 9), Vδ2-T-Exos expressed appreciable levels of cytolytic molecules (FasL, TRAIL), activating receptor (NKG2D), chemokine receptor (CCR5), antigen-presenting molecules (HLA-A/B/C; HLA-DR/DP/DQ) and costimulatory molecules (CD80, CD86) (FIG. 1E-F), suggesting their great potential in cancer immunotherapy.

### Establishment of EBV-LCL In Vitro

Buffy coats of EBV-seropositive healthy subjects were obtained after informed consents and subjected to huPBMCs isolation by Ficoll-Hypaque gradient centrifugation. EBV-LCL were established as described by Xiang et al. (2014). Briefly, huPBMCs were incubated with EBV-containing supernatants derived from B95-8 or B95.8EBfaV-GFP cell line and cultured in RPMI-1640 medium supplemented with 15% FBS in the presence of cyclosporine-A.

### Expansion of Vδ2 and Generation of Vδ2-T-Exos

Vδ2-T cells were expanded followed the protocol described by Xiang et al. (2014) and Tu et al. Briefly, huPBMCs were cultured in 10% FBS supplemented RPMI-1640 medium and stimulated with 9 μg/ml PAM at day 0 and day 3. Human recombinant interleukin-2 (IL-2; Invitrogen) was added every three day from day 3 in a final concentration of 200 IU/ml. After 14 to 20 days, Vδ2-T cells (purity >95%) were transferred to exosome-free 10% FBS-RPMI medium, in the presence of 9 μg/ml PAM and 500 IU/ml IL-2. The conditioned medium was collected after 48 h and subjected to exosome isolation.

### Vδ2-T-Exos Isolation and Characterization

Exosomes were isolated by differential ultracentrifugation at 4° C. Conditioned medium was first centrifuged at 300×g for 10 min to pellet whole cells, 2,000×g for 10 min to remove dead cells, and 10,000×g for 30 min to discard cell debris. The supernatant was then passed through 0.22-μm syringe filter and followed by ultracentrifugation at 100, 000×g for 70 min (SW32Ti rotor, Beckman) The pellet was resuspended in PBS and washed again at 100,000×g for 70 min Finally, the exosome-containing pellet was dissolved in PBS. For analysis of transmission electron microscopy, exosomes were fixed by 2% paraformaldehyde and placed on formvar-carbon-coated copper grids. The grids were then stained with 2% phosphotungstic acid and imaged using a Philips CM100 Transmission Electron Microscope (Philips, Eindhoven, Netherlands). The size distribution of exosomes was determined by dynamic light scattering (DLS) analysis using a DynaPro Plate Reader (Wyatt Technology, CA, USA). For western blot analysis, proteins from cellular lysates or exosomes were obtained by lysis in RIPA buffer in the presence of protease inhibitor Cocktail and separated by SDS-electrophoresis on 8-12% gels. Subsequently, proteins were transferred onto a nitrocellulose membrane and blocked with 5% nonfat milk. The membranes were then incubated with anti-CD63, anti-CD81, anti-TSG101, anti-Alix and anti-GRP94 antibodies overnight, respectively (Abcam, Cambridge, UK). Following incubation with corresponding secondary HRP-conjugated antibodies, the chemiluminescence signals were detected by using Immobilon Classico Western HRP substrate (Millipore, MA, USA). For FACS analysis, exosomes were conjugated with 4-μm aldehyde/sulfate latex beads by overnight incubation. The exosome-bound beads were incubated with glycine to block remaining binding sites and stained with the following fluorescent-labeled antibodies and corresponding matched isotype controls: CD63, TCR-γδ, CD4, CD8, CD19, NKG2D, FasL, TRAIL, CCR5, HLA-A/B/C, HLA-DR/DP/DQ, CD80, CD86 (Biolegend, CA, USA). Data acquisition was conducted on BD LSR II Flow cytometer (BD Biosciences, CA, USA). Moreover, the exosome-containing pellets were further characterized using iodixanol gradient centrifugation as described by Lobb et al. For cell treatment, 10 μg (unless specified otherwise) exosomes were used based on protein concentration determined by a Pierce BCA protein assay kit (Pierce). In some experiments, Vδ2-T-Exos were preincubated for 30 min with the following antibodies or corresponding matched isotype controls: anti-FasL, anti-TRAIL, anti-NKG2D, anti-TCR-γδ, anti-HLA-DR/DP/DQ, anti-HLA-A/B/C, anti-CD86 (Biolegend) and washed by ultracentrifugation to remove non-bound antibody.

Interaction of Vδ2-T-Exos with Recipient Cells

Vδ2-T-Exos were labeled with Dil or CFSE fluorescence followed the manufacturer's instruction to monitor their interaction with recipient cells. After staining with the fluorescent dyes, exosomes were washed twice with PBS by being re-centrifuged at 100,000 g for 70 min to remove excess dyes. Finally, the fluorescence-labeled exosomes were resuspended in PBS for further use. In some experiments, pellets were isolated from non-conditioned Exos-free medium using differential ultracentrifugation and labeled as described above of Vδ2-T-Exos to serve as control. To determine the cellular internalization of Vδ2-T-Exos in recipient cells, Dil-labeled Exos were incubated with allogeneic EBV-LCL cells (1×10⁵). After 18 h, the incubated cells were fixed with 4% paraformaldehyde and stained with DAPI. Confocal images were obtained by LSM710 (Zeiss, Oberkochen, German). To evaluate the uptake efficacy of Vδ2-T-Exos, CFSE⁺ cells were determined after 18 h exposure to CFSE-labeled Exos using BD LSR II Flow cytometer. In some experiments, CFSE-labeled Exos were preincubated with neutralizing anti-TCR-γδ, anti-NKG2D antibodies or corresponding isotype controls (Biolegend) for 30 min prior to be incubated with recipient cells. To determine whether Vδ2-T-Exos can target tumor site in vivo, DiR-labeled Vδ2-T-Exos were injected i.p. into EBV-induced B-cell lymphoma bearing Rag2^{-/-}γc^{-/-} mice. The accumulation of DiR-labeled Vδ2-T-Exos in tumor tissue was detected using an IVIS Spectrum in vivo imaging system (Caliper Life Sciences, Hopkinton, USA).

### Cell Apoptosis Assay

To investigate the cytolytic activities of Vδ2-T-Exos, EBV-LCL (1×10⁵) were treated with increasing amounts of Vδ2-T-Exos. Autologous normal B cells received same treatment were used as control. The apoptosis of treated cells was measured after 18 h using an Annexin V Apoptosis Detection Kit (BioLegend). In some experiments, Vδ2-T-Exos were preincubated with neutralizing anti-FasL, anti-TRAIL antibodies or corresponding isotype control before addition to EBV-LCL. Apoptosis inhibition was calculated as the percent of inhibition relative to that in the group without any treatment. In some experiments, activated caspase-3 was detected in permeabilized EBV-LCL after 4 h exposure to Vδ2-T-Exos using an anti-active-caspase-3 monoclonal antibody (BD Pharmingen, California, USA).

### Chemotaxis Assay

The chemotactic activity of CD3 T cells was determined using a transwell System (5.0 μm-pore size; Corning Costar) as described by Xiang et al. Purified CD3 T cells were treated with Vδ2-T-Exos or PBS for 48 h and harvested. The Vδ2-T-Exos-pretreated CD3 T cells were then preincubated with neutralizing anti-CCR5 antibody (20 mg/ml; clone 2D7, BD) or corresponding isotype control for 30 min and added in the upper chamber. The PBS-pretreated CD3 T cells without any preincubation were used as control. EBV-LCL derived supernatants were harvested after 24 h culture in serum-free RPMI 1640 medium and added into the lower chamber. 4 h later, the migrated CD3 cells to the lower chamber were counted using counting beads (Molecular Probes™, USA) with detection on flow cytometry. The migration of CD3 T cells in control group was set to 100% and the migration of other groups were calculated as a percentage relative to the control group.

### T-Cell Proliferation and Cytokine Secretion Assay

CD3 T cells were negatively isolated by Pan T Cell isolation kit (Miltenyi Biotec). 2×10⁵ CD3 T cells were treated with increasing amounts of autologous or allogeneic Vδ2-T-Exos. For proliferation assay, the T cells were pre-stained with CFSE (Sigma-Aldrich) according to manufacturer's instruction. After 7 days cultures, T cell proliferation was determined by flow cytometry. Before intracellular cytokine staining, the cells were re-stimulated with 100 ng/ml phorbol myristate acetate (Sigma-Aldrich), 1 μg/ml ionomycin (Sigma-Aldrich) and 10 μg/ml brefeldin A (BFA, Sigma-Aldrich) for 6 h. Cells were collected and stained for surface markers of CD4, CD8 and subjected to intracellular staining of IFN-γ. In some experiments, Vδ2-T-Exos were preincubated with neutralizing anti-HLA-A/B/C, anti-HLA-DR/DP/DQ and anti-CD86 antibodies or corresponding isotype control before addition to T cells. In some experiment, EBV-specific cytotoxic T lymphocytes (EBV-CTLs) were selected from EBV-seropositive huPBMCs using a CD137 microbeads Kit (MiltenyiBiotec, USA) after 24 h stimulation by LMP2a or EBNA1 peptide pool (MiltenyiBiotec, USA). The selected cells were treated with allogeneic Vδ2-T-Exos or PBS and cultured in the presence of 100 IU/ml IL-2. Medium were replaced every 3 days with fresh IL-2 containing medium, as well as Vδ2-T-Exos or PBS treatment. After two weeks, the cells were challenged with EBNA1 or LMP2a peptide pool for 6 h, with addition of BFA 2 h later. The EBV-CTLs were detected on flow cytometry by staining of surface markers CD4, CD8 and subjected to intracellular staining of IFN-γ. The cell numbers were counted together using counting beads (Molecular Probes™, USA).

Establishment of EBV-Induced B-Cell Lymphoma Model and Treatment of EBV-Induced B-Cell Lymphomas in Rag2^{-/-}γc^{-/-} and Humanized Mice

Rag2^{-/-}γc^{-/-} mice were cultivated in the Laboratory Animal Unit of the University of Hong Kong. Humanized mice were established from 4 to 5-week-old Rag2^{-/-}γc^{-/-} mice with reconstitution of EBV-seropositive whole huPBMCs, CD4-deplected huPBMCs, or CD8-deplected huPBMCs using the method we built before. 4 weeks after huPBMCs reconstitution, these chimeric Rag2^{-/-}γc^{-/-} became stable with functional human peripheral immune system and referred to “humanized” mice. Then the humanized mice or 6 to 8-week-old Rag2^{-/-}γc^{-/-} were implanted s.c. with EGFP-expressing EBV-LCL or EBV-LCL (0.1×10⁶/mouse) to establish the EBV-induced B-cell lymphoma model. The EBV-LCL injected mice were intraperitoneally (i.p.) administrated with equivalent volume of PBS, or Vδ2-T-Exos (25 μg/mouse) at indicated time after the inoculation with EBV-LCL. For EBV-LCL injected humanized mice, the administrated Vδ2-T-Exos were autologous to the reconstituted huPBMCs unless specified. The disease signs (ruffled hair, weight loss and activities loss), tumor incidence, tumor volume and mice survival were monitored every day. Mice bearing subcutaneous tumor with diameter larger than 17 mm were sacrificed according to the regulation in Laboratory Animal Unit of the University of Hong Kong and counted as dying. Otherwise, mice were followed up for 100 days before sacrificed. The tumors and organs were reserved and subjected to histological and immunohistochemical evaluation.

### Histological and Immunohistochemical Analysis

Tumor tissues were fixed with 10% formalin and embedded in paraffin for sectioning. The sections were subjected to hematoxylin & eosin, situ hybridization, immunohistochemistry and immunofluorescence staining. EB-encoded small RNAs type 1 and 2 (EBER-1/2) was detected by situ hybridization using a DIG-HRP REMBRANDT EBER ISH kit (Panpath, The Netherlands). Ki67 was detected by immunohistochemistry using anti-human Ki67 antibody (Abcam, UK) and visualized by a diaminobenzidine detection kit (Maixin, China). The infiltration of human T cells in tumor tissue was determined by immunofluorescence using anti-human CD3 antibody and imaged by a LSM 710 Confocal Microscopy (Zeiss, Germany)

### Flow Cytometric Analysis

Surface staining of cells was performed using the following antibodies: anti-CD63 (H5C6), anti-CD3 (HIT3a), anti-CD4 (RPA-T4), anti-CD8 (SKI), anti-CD19 (HIB19), anti-TCR-γδ (B6), anti-HLA-DQ/DP/DQ (Tü39), anti-HLA-A/B/C (W6/32), anti-CD80 (2D10), anti-CD86 (GL-1), anti-CD69 (FN50), anti-TRAIL (RIK-2), anti-FasL (NOK-1), anti-Fas (DX2), anti-DR5 (DJR2-4), anti-MICA/B (6D4), anti-NKG2D (1D11), and anti-CCR5 (2D7). For the intracellular staining, cells were fixed, permeabilized and followed by staining with anti-IFN-γ (B27) and anti-active caspase 3 (C92-605) antibodies (BD, USA) or corresponding isotype controls as described before(18, 64). All samples were detected using a FACSLSR II Flow Cytometer (BD, USA) and analyzed by FlowJo software (Tree Star, USA).

### Statistics

Data are expressed as means±SEM. The difference in fluorescence intensity, cell apoptosis, proliferation, cytokine expression and tumor volume were compared by using paired or unpaired student t-test. The tumor incidence and mice survival among different groups was compared using Kaplan-Meier log-rank test. Two-tailed test was used for all analyses. p<0.05 was regarded as significant.

All patents, patent applications, provisional applications, and publications referred to or cited herein are incorporated by reference in their entirety, including all figures and tables, to the extent they are not inconsistent with the explicit teachings of this specification.

Following are examples that illustrate procedures for practicing the invention. These examples should not be construed as limiting. All percentages are by weight and all solvent mixture proportions are by volume unless otherwise noted.

### Example 1— Vδ2-T-Exos Target EBV-Induced B-Cell Lymphomas

To determine the interaction of Vδ2-T-Exos with the tumor cells, Vδ2-T-Exos were labeled with Dil or CFSE and then added into the culture medium of EBV-transformed B lymphoblastoid cell lines (EBV-LCL) for 18 hours. Pellets isolated from non-conditioned exosome-free medium by differential ultracentrifugation were served as controls. Confocal microscopy demonstrated that Vδ2-T-Exos could be taken by EBV-LCL (FIG. 2A). To examine whether Vδ2-T-Exos can accumulate in tumor site in vivo, EBV-induced B-cell lymphoma were established in Rag2^{-/-}γc^{-/-} immunodeficient mice by subcutaneous (s.c.) inoculation of EGFP⁺ EBV-LCL as described by Xiang et al. (2014). DiR-labeled Vδ2-T-Exos or their controls were intraperitoneally (i.p.) injected into EGFP⁺ EBV-induced B-cell lymphoma bearing mice. After 3 and 24 hours, the accumulation of Vδ2-T-Exos in tumor tissues was tested using an in vivo imaging system and showed that Vδ2-T-Exos specifically accumulated in tumor tissues in vivo, compared with the controls (FIG. 2B).

To further evaluate the interaction of Vδ2-T-Exos with EBV-induced B-cell lymphoma, EBV-LCL or autologous normal B cells were incubated with CFSE-labeled Vδ2-T-Exos or the controls. Flow cytometry analysis found that all EBV-LCL became CFSE positive after treatment with CFSE-labeled Vδ2-T-Exos (FIG. 2C) Importantly, the uptake efficacy of Vδ2-T-Exos by EBV-LCL was significantly higher than that by autologous normal B cells (FIG. 2C), suggesting that Vδ2-T-Exos could target EBV-LCL. Liposome is one kind of nanoparticles that has nanospherical membrane-type structure with a lipid biolayer and shares similar physical characteristics with exosomes. Thus, liposomes were used to treat EBV-LCL or autologous normal B cells, to determine whether the different uptake efficacy of Vδ2-T-Exos between EBV-LCL and autologous normal B cells was due to the non-specific binding activity of nanoparticles. Intriguingly, no significant differences of liposome uptake efficiency were observed between EBV-LCL and autologous normal B cells (FIG. 2D), confirming that the targeting of Vδ2-T-Exos to EBV-LCL was not due to their non-specific binding Importantly, the targeting of Vδ2-T-Exos to EBV-LCL was dependent on the interaction of Vδ2-T-Exos carried NKG2D and its ligands MICA/B which constitutively expressed on EBV-LCL (FIG. 11), because blockade of Vδ2-T-Exos carried NKG2D by anti-NKG2D neutralizing mAb significantly inhibited the targeting of Vδ2-T-Exos on EBV-LCL (FIG. 2E). In contrast, blockade of Vδ2-T-Exos carried TCR-γδ could not inhibit the targeting of Vδ2-T-Exos on EBV-LCL (FIG. 2F), indicating the targeting of Vδ2-T-Exos to EBV-LCL was not dependent on Vδ2-T-Exos carried TCR-γδ. Taken together, these results demonstrated that the Vδ2-T-Exos could target EBV-induced B-cell lymphoma.

### Example 2— a Vδ2-T-Exos Induce EBV-LCL Apoptosis

To determine whether Vδ2-T-Exos can induce EBV-LCL apoptosis, EBV-LCL or autologous normal B cells were treated with different concentrations of Vδ2-T-Exos for 18 hours. As shown in FIG. 3A, Vδ2-T-Exos induced EBV-LCL apoptosis in a dose-dependent manner, but they had no such effect on autologous normal B cells. The apoptosis was mainly induced by the exosomes fraction 6, 7 and 8 (FIG. 8C). The apoptosis of EBV-LCL induced by Vδ2-T-Exos was caspase-dependent as evidenced by the increased active caspase-3 expression in Vδ2-T-Exos treated EBV-LCL (FIG. 3B). EBV-LCL had higher levels of surface Fas and TRAIL receptor 2 (death-inducing receptor, DR5) expressions than autologous normal B cells (FIG. 3C). Vδ2-T-Exos also carried robust death-inducing ligands (FasL, TRAIL) (FIG. 1E). Blockade of Fas/FasL or TRAIL/DR5 pathway by using neutralizing anti-FasL or anti-TRAIL monoclonal antibody significantly inhibited EBV-LCL apoptosis induced by Vδ2-T-Exos (FIG. 3D), indicating that Vδ2-T-Exos-induced EBV-LCL apoptosis was mediated, at least in part, by Fas/FasL and TRAIL/DR5 pathways.

### Example 3— Vδ2-T-Exos Control EBV-Induced B-Cell Lymphomas in Rag2^{-/-}γc^{-/-} Mice

EGFP⁺ EBV-LCL were used to monitor EBV-induced B-cell lymphoma growth in vivo, and the EBV-induced B-cell lymphoma model was further established in Rag2^{-/-}γc^{-/-} mice after inoculation s.c. of EGFP⁺ EBV-LCL (FIG. 4A) as described by Xiang et al. (2014). Vδ2-T-Exos were then administered i.p. into Rag2^{-/-}γc^{-/-} mice weekly from day 0 for up to ten doses (FIG. 4A). After inoculation of EGFP⁺ EBV-LCL, rapid development of solid tumors was detected in all PBS-treated mice by in vivo imaging (FIGS. 4B and C). These tumors were immunoblastic lymphomas and derived from human B cells as evidenced by positive staining for human CD20. High expression of small EBV-encoded RNAs type 1/2 (EBER-1/2) indicated that these tumors were EBV-associated lymphomas (FIG. 4D). Compared with PBS treated group, Vδ2-T-Exos treatment significantly reduced tumor incidence (FIG. 4C). The tumor growth was also significantly inhibited in Vδ2-T-Exos-treated mice as evidenced by in vivo imaging analysis (FIG. 4B) and tumor volume (FIG. 4E). Most importantly, Vδ2-T-Exos treatment significantly prolonged survival of EBV-induced B-cell lymphoma-grafted immunodeficient mice (FIG. 4F). Histological and immunophenotypic analysis of residual tumors found that there were fewer Ki-67 positive cells within EBV-induced B-cell lymphomas in Vδ2-T-Exos-treated mice than those in PBS-treated mice, indicating that the residual tumor cells in Vδ2-T-Exos-treated mice had lower proliferative capacity than that in PBS-treated mice (FIG. 4D). Taken together, these data suggested that Vδ2-T-Exos can control the development of EBV-induced B-cell lymphomas in Rag2^{-/-}γc^{-/-} mice.

To further determine whether Vδ2-T-Exos have therapeutic effects on EBV-induced B-cell lymphomas, EGFP⁺ EBV-LCL were implanted into Rag2^{-/-}γc^{-/-} mice (FIG. 4G). After 14 days, mice that had developed subcutaneous tumors as detected by in vivo imaging were randomly divided into two groups (FIG. 4H). There was no significant difference of fluorescent density from tumor cells between the two groups (FIG. 4H). One group of the tumor-bearing mice received Vδ2-T-Exos treatment weekly from day 14 to day 77, while another group of the tumor-bearing mice received PBS as the control (FIG. 4G). The PBS-treated mice had subcutaneous tumors with progressive growth, and all died within 56 days after EBV-LCL implantation (FIG. 4I) Importantly, Vδ2-T-Exos treatment significantly limited tumor growth (FIG. 4J) and improved mice survival (FIG. 4I). Histological and immunohistochemical analysis indicated that these residual tumors were EBV-associated as they expressed EBER-1/2 (FIG. 4K). Moreover, there were extremely numerous Ki-67 positive cells in tumor tissues from PBS-treated mice, while there were only a few of Ki-67 positive cells in tumor tissues in Vδ2-T-Exos-treated mice (FIG. 4K). These results indicated that Vδ2-T-Exos can constrain tumor growth in EBV-induced B-cell lymphoma-bearing immunodeficient mice.

### Example 4— Vδ2-T-Exos Control the Development of EBV-Induced B-Cell Lymphomas in Humanized Mice

Humanized mice with stable reconstitution of functional human peripheral blood mononuclear cells (huPBMCs) were generated as described by Xiang et al. (2014) and Tu et al. EBV-induced B-cell lymphoma model was then established by s.c. inoculation of autologous EBV-LCL in humanized mice as described by Xiang et al. (2014). After the inoculation of EBV-LCL, Vδ2-T-Exos were injected i.p. into humanized mice weekly from day 0 to day 63 (FIG. 5A). Similar to that in Rag2^{-/-}γc^{-/-} mice, all PBS-treated humanized mice developed subcutaneous solid tumors within 21 days after EBV-LCL inoculation (FIG. 5B). The tumor incidence in Vδ2-T-Exos-treated humanized mice was significantly lower than that in PBS-treated humanized mice during 100 days of observation (FIG. 5B). Vδ2-T-Exos treatment efficiently inhibited the tumor growth in humanized mice (FIG. 5C). Most importantly, Vδ2-T-Exos treatment significantly prolonged the survival of humanized mice (FIG. 5D). In PBS-treated group, all humanized mice died within 56 days after EBV-LCL implantation. In contrast, only 3 out of 8 Vδ2-T-Exos-treated mice died and the rest of mice were still alive during 100 days of observation (FIG. 5D). Consistently, these tumors were positive with EBER1/2 (FIG. 5E). The residual tumor tissues in Vδ2-T-Exos-treated humanized mice had less ki-67 positive cells than PBS-treated mice (FIG. 5E), indicating that Vδ2-T-Exos could suppress the proliferative capability of tumor cells in vivo. These data demonstrated that Vδ2-T-Exos can efficiently control the development of EBV-induced B-cell lymphomas in humanized mice.

### Example 5— Allogeneic Vδ2-T-Exos have Better Therapeutic Effect than Autologous Vδ2-T-Exos on EBV-Induced B-Cell Lymphomas in Humanized Mice

As most cancer patients are immunocompromised, it is very difficult to expand their own Vδ2-T cells in vitro and prepare enough Vδ2-T-Exos for clinical use. Therefore, therapeutic effects were compared between autologous and allogeneic Vδ2-T-Exos on EBV-induced B-cell lymphomas in humanized mice (FIG. 5F). As shown in FIG. 5G, both autologous and allogeneic Vδ2-T-Exos could control the development of EBV-induced B-cell lymphomas in humanized mice when compared with the control Importantly, allogeneic Vδ2-T-Exos were more potent than autologous Vδ2-T-Exos to control the development of EBV-induced B-cell lymphomas in humanized mice, in terms of tumor incidence (FIG. 5G), tumor growth (FIG. 5H) and mice survival (FIG. 5I). Moreover, the proliferative capability of tumor cells was significantly lower in allogeneic Vδ2-T-Exos-treated humanized mice than that in autologous Vδ2-T-Exos-treated mice, as evidenced by the decreased Ki-67 expression in residual tumor in allogeneic Vδ2-T-Exos-treated mice (FIG. 5J).

Immunofluorescent analysis found that more CD3 T cells infiltrated into tumor tissues after treated with allogeneic Vδ2-T-Exos when compared with those treated with autologous Vδ2-T-Exos or PBS (FIG. 5K), suggesting allogeneic Vδ2-T-Exos might boost host T cells-mediated antitumor responses. To confirm this, the therapeutic effects of allogeneic Vδ2-T-Exos on EBV-induced B-cell lymphomas were compared between humanized mice and Rag2^{-/-}γc^{-/-} mice. Treatment with allogeneic Vδ2-T-Exos in humanized mice was more effective than that in Rag2^{-/-}γc^{-/-} mice to reduce tumor incidence (FIG. 5G) and tumor growth (FIG. 5H), and prolong mice survival (FIG. 5I). As the only difference between humanized mice and Rag2^{-/-}γc^{-/-} mice was that humanized mice had reconstituted functional human immune cells, these results suggested that human immune cells, especially T cells, were probably involved in Vδ2-T-Exos-induced antitumor activities in humanized mice.

### Example 6— Vδ2-T-Exos Induce CD4 and CD8 T Cell-Mediated Antitumor Immunity

Allogeneic Vδ2-T-Exos treatment increased the infiltration of CD3 T cells into EBV-induced B-cell lymphoma tissues in humanized mice (FIG. 5K). Whether allogeneic Vδ2-T-Exos could induce CD4 and CD8 T cell-mediated antitumor immunity against EBV-induced B-cell lymphomas was tested. Both CD4 and CD8 T cells could interact with Vδ2-T-Exos as demonstrated by the increased CFSE signal in both CD4 and CD8 T cells after exposure to CFSE-labeled Vδ2-T-Exos (FIGS. 6A and B). Interestingly, allogeneic Vδ2-T-Exos significantly increased the expression of CCR5 in both CD4 and CD8 T cells compared with control group (FIGS. 6C and D). In a transwell chemotaxis system, Vδ2-T-Exos treatment significantly increased the migration of T cells towards EBV-LCL, and this migration could be significantly inhibited by anti-CCR5 blocking antibody (FIG. 5E). These results demonstrated that Vδ2-T-Exos could increase the infiltration of T cells into EBV-induced B-cell lymphoma tissues by upregulating the expression of CCR5 on T cells.

To compare the effects of autologous and allogeneic Vδ2-T-Exos on T cell responses, the proliferation and IFN-γ production in T cells were determined. As shown in FIGS. 6F-6I, both autologous and allogeneic Vδ2-T-Exos could induce cell proliferation and IFN-γ production in CD4 and CD8 T cells. Blockade of HLA-DR/DP/DQ or CD86 pathway with neutralizing anti-LA/DR/DP/DQ or anti-CD86 monoclonal antibody significantly inhibited Vδ2-T-Exos-induced cell proliferation and IFN-γ production in CD4 T cells (FIGS. 10A, 10B, 10E, and 10F), while blockade of HLA-A/B/C or CD86 pathway with their neutralizing monoclonal antibody did not significantly affect Vδ2-T-Exos-induced cell proliferation and IFN-γ production in CD8 T cells (FIGS. 10C, 10D, 10G, and 10H), suggesting the recognitions of HLA molecules and CD86 are more important for Vδ2-T-Exos-induced CD4 cell responses than Vδ2-T-Exos-induced CD8 cell responses Importantly, allogeneic Vδ2-T-Exos were more potent to induce cell proliferation and IFN-γ production in CD4 and CD8 T cells than autologous Vδ2-T-Exos (FIG. 6F-I). Thus, allogeneic Vδ2-T-Exos induced more potent T cell responses than autologous Vδ2-T-Exos in vitro.

EBV-seropositive healthy individuals carry significant population of EBV-specific T cells, which play important roles in the control of EBV-induced lymphoma. The effect of Vδ2-T-Exos on EBV-specific T cells was determined. As shown in FIGS. 6J-6K, allogeneic Vδ2-T-Exos significantly promoted the expansions of EBV EBNA1-specific CD4 and LMP2a-specific CD8 T cell clones compared with PBS, indicating that Vδ2-T-Exos could also promote the pre-existing tumor antigen-specific T cell expansion and enhance their therapeutic efficacy against EBV-induced B-cell lymphoma.

### Example 7—CD4 and CD8 T Cells are Involved in Vδ2-T-Exos-Induced Antitumor Immunity in Humanized Mice

To further elucidate the roles of CD4 and CD8 T cells in allogeneic Vδ2-T-Exos-mediated antitumor immunity in vivo, humanized mice reconstituted with EBV-seropositive whole huPBMCs, CD4-T-cell-depleted huPBMCs or CD8-T-cell-depleted huPBMCs were established. Allogeneic Vδ2-T-Exos were then injected i.p. into humanized mice weekly after the inoculation of EBV-LCL from day 0 to day 63 (FIG. 7A). Compared with that in humanized mice reconstituted with whole huPBMCs, Vδ2-T-Exos-mediated antitumor efficacy was significantly reduced in humanized mice reconstituted with either CD4-T-cell-depleted huPBMCs or CD8-T-cell-depleted huPBMCs, in terms of tumor incidence (FIG. 7B), tumor volume (FIG. 7C) and mice survival (FIG. 7D). In addition, there was no difference about Vδ2-T-Exos-mediated antitumor efficacy between CD4-T-cell-depleted huPBMCs and CD8-T-cell-depleted huPBMCs reconstituted humanized mice (FIG. 7). These results indicate that Vδ2-T-Exos induced CD4 and CD8 T cell-mediated antitumor immunity against EBV-induced B-cell lymphomas in humanized mice.

### Example 8— Optimized Protocol to Generate More Vδ2-T-Exos with Enhanced Antitumor Activities

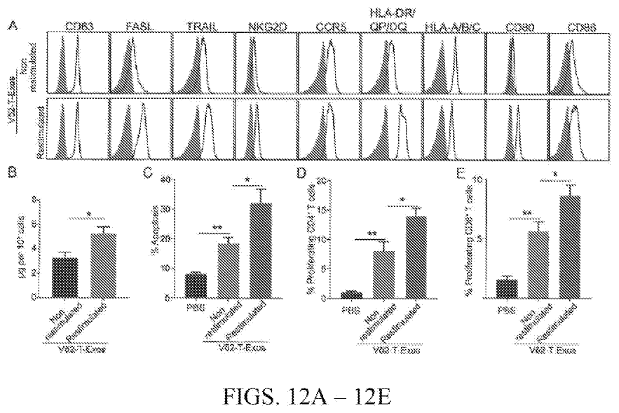

Human peripheral blood was slowly loaded onto Ficoll-Hypaque (Lymphoprep, Fresenius Kabi Norge AS, Oslo, Norway) for gradient centrifugation at 1,000×g for 20 min without brake. After centrifugation, human peripheral blood mononuclear cells (huPBMCs) were isolated carefully from the interphase between plasma layer and Ficoll-Hypaque layer. Isolated PBMCs were washed with PBS twice and centrifuged at 300×g for 10 min to remove the resident Ficoll-Hypaque. huPMBCs were cultured in RPMI-1640 medium supplemented with 10% fetal bovine serum (FBS, Invitrogen). PAM was added at day 0 and day 3 to a concentration of 9 ng/ml. Recombinant human IL-2 (rhIL-2, Invitrogen) was added to a final concentration of 200 IU/ml every 3 days from day 3. After 14-20 days, the expanded Vδ2-T cells were conditioned by re-stimulating with 9 ng/ml PAM plus 400 IU/ml IL-2 for 48 h in exosome-free 10% FBS-RPMI medium. After conditioning, the exosome-containing supernatant was harvested and centrifuged at 300×g for 10 minutes to pellet whole cells, 2,000×g for 10 minutes to remove dead cells, and 10,000×g for 30 minutes to discard cell debris. The supernatant was then passed through 0.22 nm syringe filter and followed by ultracentrifugation at 100,000×g for 70 minutes. The pellet is resuspended in PBS and washed again at 100,000×g for 70 minutes. Finally, the exosome-containing pellet is dissolved in PBS and either used immediately or stored at −80° C. FIG. 12 shows the phenotype (A), production (B) and functions (C-E) of exosomes derived from Vδ2-T cells with or without re-stimulation during conditioning.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### REFERENCES

1. R. Kuppers, B cells under influence: transformation of B cells by Epstein-Ban virus. Nat Rev Immunol 3, 801-812 (2003).
2. L. S. Young, L. F. Yap, P. G. Murray, Epstein-Ban virus: more than 50 years old and still providing surprises. Nat Rev Cancer 16, 789-802 (2016).
3. J. L. Crombie, A. S. LaCasce, Epstein Ban Virus Associated B-Cell Lymphomas and Iatrogenic Lymphoproliferative Disorders. Front Oncol 9, 109 (2019).
4. V. R. Dharnidharka, A. C. Webster, O. M. Martinez, J. K. Preiksaitis, V. Leblond, S. Choquet, Post-transplant lymphoproliferative disorders. Nat Rev Dis Primers 2, 15088 (2016).
5. R. Dolcetti, B lymphocytes and Epstein-Ban virus: the lesson of post-transplant lymphoproliferative disorders. Autoimmun Rev 7, 96-101 (2007).
6. V. R. Dharnidharka, T. Mohanakumar, New approaches to treating B-cell cancers induced by Epstein-Ban virus. N Engl J Med 372, 569-571 (2015).
7. J. A. Kanakry, R. F. Ambinder, EBV-related lymphomas: new approaches to treatment. Curr Treat Options Oncol 14, 224-236 (2013).
8. C. M. Bollard, C. M. Rooney, H. E. Heslop, T-cell therapy in the treatment of post-transplant lymphoproliferative disease. Nat Rev Clin Oncol 9, 510-519 (2012).
9. S. Beetz, D. Wesch, L. Marischen, S. Welte, H. H. Oberg, D. Kabelitz, Innate immune functions of human gammadelta T cells. Immunobiology 213, 173-182 (2008).
10. W. K. Born, C. L. Reardon, R. L. O'Brien, The function of gammadelta T cells in innate immunity. Curr Opin Immunol 18, 31-38 (2006).
11. S. R. Carding, P. J. Egan, Gammadelta T cells: functional plasticity and heterogeneity. Nat Rev Immunol 2, 336-345 (2002).
12. D. Hannani, Y. Ma, T. Yamazaki, J. Dechanet-Merville, G. Kroemer, L. Zitvogel, Harnessing gammadelta T cells in anticancer immunotherapy. Trends Immunol 33, 199-206 (2012).
13. D. Kabelitz, D. Wesch, W. He, Perspectives of gammadelta T cells in tumor immunology. Cancer Res 67, 5-8 (2007).
14. E. J. Adams, S. Gu, A. M. Luoma, Human gamma delta T cells: Evolution and ligand recognition. Cell Immunol 296, 31-40 (2015).
15. H. J. Gober, M. Kistowska, L. Angman, P. Jeno, L. Mori, G. De Libero, Human T cell receptor gammadelta cells recognize endogenous mevalonate metabolites in tumor cells. J Exp Med 197, 163-168 (2003).
16. M. Bonneville, E. Scotet, Human Vgamma9Vdelta2 T cells: promising new leads for immunotherapy of infections and tumors. Curr Opin Immunol 18, 539-546 (2006).
17. H. Li, Z. Xiang, T. Feng, J. Li, Y. Liu, Y. Fan, Q. Lu, Z. Yin, M. Yu, C. Shen, W. Tu, Human Vgamma9Vdelta2-T cells efficiently kill influenza virus-infected lung alveolar epithelial cells. Cell Mol Immunol 10, 159-164 (2013).
18. Z. Xiang, Y. Liu, J. Zheng, M. Liu, A. Lv, Y. Gao, H. Hu, K. T. Lam, G. C. Chan, Y. Yang, H. Chen, G. S. Tsao, M. Bonneville, Y. L. Lau, W. Tu, Targeted activation of human Vgamma9Vdelta2-T cells controls epstein-barr virus-induced B cell lymphoproliferative disease. Cancer Cell 26, 565-576 (2014).
19. Z. Xiang, W. Tu, Dual Face of Vgamma9Vdelta2-T Cells in Tumor Immunology: Anti-versus Pro-Tumoral Activities. Front Immunol 8, 1041 (2017).
20. H. Sicard, S. Ingoure, B. Luciani, C. Serraz, J. J. Fournie, M. Bonneville, J. Tiollier, F. Romagne, In vivo immunomanipulation of V gamma 9V delta 2 T cells with a synthetic phosphoantigen in a preclinical nonhuman primate model. J Immunol 175, 5471-5480 (2005).
21. J. M. Pitt, A. Marabelle, A. Eggermont, J. C. Soria, G. Kroemer, L. Zitvogel, Targeting the tumor microenvironment: removing obstruction to anticancer immune responses and immunotherapy. Ann Oncol 27, 1482-1492 (2016).
22. G. Raposo, W. Stoorvogel, Extracellular vesicles: exosomes, microvesicles, and friends. J Cell Biol 200, 373-383 (2013).
23. B. M. Bell, I. D. Kirk, S. Hiltbrunner, S. Gabrielsson, J. J. Bultema, Designer exosomes as next-generation cancer immunotherapy. Nanomedicine 12, 163-169 (2016).
24. N. L. Syn, L. Wang, E. K. Chow, C. T. Lim, B. C. Goh, Exosomes in Cancer Nanomedicine and Immunotherapy: Prospects and Challenges. Trends Biotechnol 35, 665-676 (2017).
25. J. M. Pitt, F. Andre, S. Amigorena, J. C. Soria, A. Eggermont, G. Kroemer, L. Zitvogel, Dendritic cell-derived exosomes for cancer therapy. J Clin Invest 126, 1224-1232 (2016).
26. L. Lugini, S. Cecchetti, V. Huber, F. Luciani, G. Macchia, F. Spadaro, L. Paris, L. Abalsamo, M. Colone, A. Molinari, F. Podo, L. Rivoltini, C. Ramoni, S. Fais, Immune surveillance properties of human NK cell-derived exosomes. J Immunol 189, 2833-2842 (2012).
27. L. Zhu, S. Kalimuthu, P. Gangadaran, J. M. Oh, H. W. Lee, S. H. Baek, S. Y. Jeong, S. W. Lee, J. Lee, B. C. Ahn, Exosomes Derived From Natural Killer Cells Exert Therapeutic Effect in Melanoma. Theranostics 7, 2732-2745 (2017).
28. R. J. Lobb, M. Becker, S. W. Wen, C. S. Wong, A. P. Wiegmans, A. Leimgruber, A. Moller, Optimized exosome isolation protocol for cell culture supernatant and human plasma. J Extracell Vesicles 4, 27031 (2015).
29. S. E. Emam, H. Ando, A. S. A. Lila, T. Shimizu, K. Okuhira, Y. Ishima, M. A. Mandy, F. S. Ghazy, I. Sagawa, T. Ishida, Liposome co-incubation with cancer cells secreted exosomes (extracellular vesicles) with different proteins expressions and different uptake pathways. Sci Rep 8, 14493 (2018).
30. W. Tu, J. Zheng, Y. Liu, S. F. Sia, M. Liu, G. Qin, I. H. Ng, Z. Xiang, K. T. Lam, J. S. Peiris, Y. L. Lau, The aminobisphosphonate pamidronate controls influenza pathogenesis by expanding a gammadelta T cell population in humanized mice. J Exp Med 208, 1511-1522 (2011).
31. Q. Sun, R. Burton, V. Reddy, K. G. Lucas, Safety of allogeneic Epstein-Ban virus (EBV)-specific cytotoxic T lymphocytes for patients with refractory EBV-related lymphoma. Br J Haematol 118, 799-808 (2002).
32. C. M. Bollard, S. Gottschalk, M. Helen Huls, A. M. Leen, A. P. Gee, C. M. Rooney, Good manufacturing practice-grade cytotoxic T lymphocytes specific for latent membrane proteins (LMP)-1 and LMP2 for patients with Epstein-Ban virus-associated lymphoma. Cytotherapy 13, 518-522 (2011).
33. E. Doubrovina, B. Oflaz-Sozmen, S. E. Prockop, N. A. Kernan, S. Abramson, J. Teruya-Feldstein, C. Hedvat, J. F. Chou, G. Heller, J. N. Barker, F. Boulad, H. Castro-Malaspina, D. George, A. Jakubowski, G. Koehne, E. B. Papadopoulos, A. Scaradavou, T. N. Small, R. Khalaf, J. W. Young, R. J. O'Reilly, Adoptive immunotherapy with unselected or EBV-specific T cells for biopsy-proven EBV+ lymphomas after allogeneic hematopoietic cell transplantation. Blood 119, 2644-2656 (2012).
34. N. N. Shah, K. Baird, C. P. Delbrook, T. A. Fleisher, M. E. Kohler, S. Rampertaap, K. Lemberg, C. K. Hurley, D. E. Kleiner, M. S. Merchant, S. Pittaluga, M. Sabatino, D. F. Stroncek, A. S. Wayne, H. Zhang, T. J. Fry, C. L. Mackall, Acute GVHD in patients receiving IL-15/4-1BBL activated NK cells following T-cell-depleted stem cell transplantation. Blood 125, 784-792 (2015).
35. A. Tan, H. De La Pena, A. M. Seifalian, The application of exosomes as a nanoscale cancer vaccine. Int J Nanomedicine 5, 889-900 (2010).
36. B. Besse, M. Charrier, V. Lapierre, E. Dansin, O. Lantz, D. Planchard, T. Le Chevalier, A. Livartoski, F. Barlesi, A. Laplanche, S. Ploix, N. Vimond, I. Peguillet, C. Thery, L. Lacroix, I. Zoernig, K. Dhodapkar, M. Dhodapkar, S. Viaud, J. C. Soria, K. S. Reiners, E. Pogge von Strandmann, F. Vely, S. Rusakiewicz, A. Eggermont, J. M. Pitt, L. Zitvogel, N. Chaput, Dendritic cell-derived exosomes as maintenance immunotherapy after first line chemotherapy in NSCLC. Oncoimmunology 5, e1071008 (2016).
37. B. Escudier, T. Dorval, N. Chaput, E Andre, M. P. Caby, S. Novault, C. Flament, C. Leboulaire, C. Borg, S. Amigorena, C. Boccaccio, C. Bonnerot, O. Dhellin, M. Movassagh, S. Piperno, C. Robert, V. Sena, N. Valente, J. B. Le Pecq, A. Spatz, O. Lantz, T. Tursz, E. Angevin, L. Zitvogel, Vaccination of metastatic melanoma patients with autologous dendritic cell (DC) derived-exosomes: results of thefirst phase I clinical trial. J Transl Med 3, 10 (2005).
38. M. A. Morse, J. Garst, T. Osada, S. Khan, A. Hobeika, T. M. Clay, N. Valente, R. Shreeniwas, M. A. Sutton, A. Delcayre, D. H. Hsu, J. B. Le Pecq, H. K. Lyerly, A phase I study of dexosome immunotherapy in patients with advanced non-small cell lung cancer. J Transl Med 3, 9 (2005).
39. J. Song, J. Huang, X. Chen, X. Teng, Z. Song, Y. Xing, M. Wang, K. Chen, Z. Wang, P. Yang, S. Hu, Donor-derived exosomes induce specific regulatory T cells to suppress immune inflammation in the allograft heart. Sci Rep 7, 20077 (2016).
40. K. V. Chow, R. M. Sutherland, Y. Zhan, A. M. Lew, Heterogeneity, functional specialization and differentiation of monocyte-derived dendritic cells. Immunol Cell Biol 95, 244-251 (2017).
41. A. A. Alexander, A. Maniar, J. S. Cummings, A. M. Hebbeler, D. H. Schulze, B. R. Gastman, C. D. Pauza, S. E. Strome, A. I. Chapoval, Isopentenyl pyrophosphate-activated CD56+{gamma} {delta} T lymphocytes display potent antitumor activity toward human squamous cell carcinoma. Clin Cancer Res 14, 4232-4240 (2008).
42. L. Kouakanou, Y. Xu, C. Peters, J. He, Y. Wu, Z. Yin, D. Kabelitz, Vitamin C promotes the proliferation and effector functions of human gammadelta T cells. Cell Mol Immunol, (2019).
43. G. Qin, H. Mao, J. Zheng, S. F. Sia, Y. Liu, P. L. Chan, K. T. Lam, J. S. Peiris, Y. L. Lau, W. Tu, Phosphoantigen-expanded human gammadelta T cells display potent cytotoxicity against monocyte-derived macrophages infected with human and avian influenza viruses. J Infect Dis 200, 858-865 (2009).
44. M. W. Khan, S. M. Curbishley, H. C. Chen, A. D. Thomas, H. Pircher, D. Mavilio, N. M. Steven, M. Eberl, B. Moser, Expanded Human Blood-Derived gammadeltaT Cells Display Potent Antigen-Presentation Functions. Front Immunol 5, 344 (2014).
45. M. Brandes, K. Willimann, B. Moser, Professional antigen-presentation function by human gammadelta T Cells. Science 309, 264-268 (2005).
46. M. S. Braza, B. Klein, Anti-tumour immunotherapy with Vgamma9Vdelta2 T lymphocytes: from the bench to the bedside. Br J Haematol 160, 123-132 (2013).
47. A. Y. Jong, C. H. Wu, J. Li, J. Sun, M. Fabbri, A. S. Wayne, R. C. Seeger, Large-scale isolation and cytotoxicity of extracellular vesicles derived from activated human natural killer cells. J Extracell Vesicles 6, 1294368 (2017).
48. L. Zitvogel, A. Regnault, A. Lozier, J. Wolfers, C. Flament, D. Tenza, P. Ricciardi-Castagnoli, G. Raposo, S. Amigorena, Eradication of established murine tumors using a novel cell-free vaccine: dendritic cell-derived exosomes. Nat Med 4, 594-600 (1998).
49. Z. Lu, B. Zuo, R. Jing, X. Gao, Q. Rao, Z. Liu, H. Qi, H. Guo, H. Yin, Dendritic cell-derived exosomes elicit tumor regression in autochthonous hepatocellular carcinoma mouse models. J Hepatol 67, 739-748 (2017).
50. I. Parolini, C. Federici, C. Raggi, L. Lugini, S. Palleschi, A. De Milito, C. Coscia, E. Iessi, M. Logozzi, A. Molinari, M. Colone, M. Tatti, M. Sargiacomo, S. Fais, Microenvironmental pH is a key factor for exosome traffic in tumor cells. J Biol Chem 284, 34211-34222 (2009).
51. J. W. Fabre, The allogeneic response and tumor immunity. Nat Med 7, 649-652 (2001).
52. N. W. Nisbet, M. Simonsen, M. Zaleski, The frequency of antigen-sensitive cells in tissue transplantation. A commentary on clonal selection. J Exp Med 129, 459-467 (1969).
53. H. A. Gjertsen, K. E. Lundin, T. Hansen, E. Thorsby, T cells specific for viral antigens presented by HLA-Dw4 recognize DR13 on allogeneic cells: a possible mechanism for induction of rejection. Transplantation proceedings 25, 70-71 (1993).
54. S. R. Burrows, R. Khanna, J. M. Burrows, D. J. Moss, An alloresponse in humans is dominated by cytotoxic T lymphocytes (CTL) cross-reactive with a single Epstein-Ban virus CTL epitope: implications for graft-versus-host disease. J Exp Med 179, 1155-1161 (1994).
55. S. Y. Li, Y. Liu, C. F. Xu, S. Shen, R. Sun, X. J. Du, J. X. Xia, Y. H. Zhu, J. Wang, Restoring anti-tumor functions of T cells via nanoparticle-mediated immune checkpoint modulation. J Control Release 231, 17-28 (2016).
56. D. A. Newton, P. M. Acierno, F. J. Brescia, E. A. Brown, S. Gattoni-Celli, Semi-allogeneic vaccines for patients with cancer and AIDS. J Immunother 25, 334-341 (2002).
57. L. D. Barber, J. A. Madrigal, Exploiting beneficial alloreactive T cells. Vox Sang 91, 20-27 (2006).
58. T. D. de Gruijl, A. J. van den Eertwegh, H. M. Pinedo, R. J. Scheper, Whole-cell cancer vaccination: from autologous to allogeneic tumor- and dendritic cell-based vaccines. Cancer Immunol Immunother 57, 1569-1577 (2008).
59. G. Qin, Y. Liu, J. Zheng, I. H. Ng, Z. Xiang, K. T. Lam, H. Mao, H. Li, J. S. Peiris, Y. L. Lau, W. Tu, Type 1 responses of human Vgamma9Vdelta2 T cells to influenza A viruses. J Virol 85, 10109-10116 (2011).
60. G. Qin, Y. Liu, J. Zheng, Z. Xiang, I. H. Ng, J. S. Malik Peiris, Y. L. Lau, W. Tu, Phenotypic and functional characterization of human gammadelta T-cell subsets in response to influenza A viruses. J Infect Dis 205, 1646-1653 (2012).
61. M. Alnaggar, Y. Xu, J. Li, J. He, J. Chen, M. Li, Q. Wu, L. Lin, Y. Liang, X. Wang, J. Li, Y. Hu, Y. Chen, K. Xu, Y. Wu, Z. Yin, Allogenic Vgamma9Vdelta2 T cell as new potential immunotherapy drug for solid tumor: a case study for cholangiocarcinoma. J Immunother Cancer 7, 36 (2019).
62. P. Speck, R. Longnecker, Epstein-Barr virus (EBV) infection visualized by EGFP expression demonstrates dependence on known mediators of EBV entry. Arch Virol 144, 1123-1137 (1999).
63. C. Thery, S. Amigorena, G. Raposo, A. Clayton, Isolation and characterization of exosomes from cell culture supernatants and biological fluids. Curr Protoc Cell Biol Chapter 3, Unit 3 22 (2006).
64. K. Ni, M. Liu, J. Zheng, L. Wen, Q. Chen, Z. Xiang, K. T. Lam, Y. Liu, G. C. Chan, Y. L. Lau, W. Tu, PD-1/PD-L1 Pathway Mediates the Alleviation of Pulmonary Fibrosis by Human Mesenchymal Stem Cells in Humanized Mice. Am J Respir Cell Mol Biol 58, 684-695 (2018).

1. A method for killing or inhibiting the growth of an EBV-infected cell, comprising contacting the EBV-infected cell with exosomes from Vδ2⁺ T cells in an amount effective to kill or inhibit the growth of the cell.

2. The method of claim 1, wherein the EBV-infected cell is an EBV-infected lymphocyte or an EBV-infected epithelial cell.

3. The method of claim 1, wherein the EBV-infected lymphocyte is an EBV-infected B-lymphocyte.

4. The method of claim 2, wherein the EBV-infected lymphocyte is an EBV-infected neoplastic B-lymphocyte.

5. The method of claim 2, wherein the EBV-infected epithelial cell is an EBV-infected neoplastic epithelial cell.

6. The method of claim 1, wherein the exosomes are isolated from Vδ2⁺ T cells that are autologous to the EBV-infected cell.

7. The method of claim 1, wherein the exosomes are isolated from Vδ2⁺ T cells that are allogeneic to the EBV-infected cell.

8. A method of treating an EBV-induced cancer, comprising administering to a subject in need thereof a therapeutically effective amount of exosomes from Vδ2⁺ T cells.

9. The method of claim 8, wherein the cancer is of lymphocytic origin or epithelial origin.

10. The method of claim 9, wherein the EBV-induced cancer of the lymphocytic origin is an EBV-induced: Burkitt lymphoma, Hodgkin's lymphoma, diffuse large B-cell lymphoma, or a lymphoproliferative disease.

11. The method of claim 9, wherein the EBV-induced cancer of the epithelial origin is an EBV-induced nasopharyngeal carcinoma (NPC) or an EBV-induced gastric cancer/carcinoma.

12. The method of claim 8, wherein the exosomes are obtained from the subject's Vδ2⁺ T cells.

13. The method of claim 12, wherein the exosomes are obtained from Vδ2⁺ T cells from the subject when the subject was known to be free of cancer.

14. The method of claim 8, wherein the exosomes are obtained Vδ2⁺ T cells from an individual who is allogeneic to the subject.

15. The method of claim 8, comprising administering the exosomes via a route selected from oral, rectal, nasal, topical, buccal, sublingual, transdermal, vaginal, intramuscular, subcutaneous, intravenous, epidural, intrathecal, and central.

16. A method of isolating Vδ2-T-Exos, comprising the steps of:
a) providing peripheral mononuclear cells (PBMCs),
b) culturing the PBMCs in a culture medium in the presence of a phosphoantigen and IL-2 for a first period of time,
c) after the first period of time, culturing the PBMCs in an exosome free culture medium in the presence of the phosphoantigen and IL-2 for a second period of time,
d) isolating the exosomes from the culture supernatant after the second period.

17. The method of claim 16, wherein the PBMCs are human PBMCs.

18. The method of claim 16, wherein the first period of time is between 14 to 20 days.

19. The method of claim 16, wherein the second period of time is 24 to 72 hours.

20. The method of claim 16, wherein the step of isolating comprises one or more of: filtration, centrifugation, and ultracentrifugation.

21. The method of claim 16, wherein the phosphoantigen is isopentenyl pyrophosphate (IPP), (E)-4-hydroxy-3-methyl-but-2-enyl-pyrophosphate (HMB-PP), bromohydrin pyrophosphate (BrHPP), Pamidronate (PAM), or any combination thereof.

22. A method for killing or inhibiting the growth of an EBV-infected cell, comprising contacting the EBV-infected cell with exosomes from Vδ2⁺ T cells isolated according to the method of claim 16 in an amount effective to kill or inhibit the growth of the cell.

23. The method of claim 22, wherein the EBV-infected cell is an EBV-infected lymphocyte or an EBV-infected epithelial cell.

24. The method of claim 22, wherein the EBV-infected lymphocyte is an EBV-infected B-lymphocyte.

25. The method of claim 23, wherein the EBV-infected lymphocyte is an EBV-infected neoplastic B-lymphocyte.

26. The method of claim 23, wherein the EBV-infected epithelial cell is an EBV-infected neoplastic epithelial cell.

27. The method of claim 22, wherein the exosomes are isolated from Vδ2⁺ T cells that are autologous to the EBV-infected cell.

28. The method of claim 22, wherein the exosomes are isolated from Vδ2⁺ T cells that are allogeneic to the EBV-infected cell.

29. A method of treating EBV-induced cancer, comprising administering to a subject in need thereof a therapeutically effective amount of exosomes from Vδ2⁺ T cells isolated according to the method of claim 16.

30. The method of claim 29, wherein the cancer is of lymphocytic origin or epithelial origin.

31. The method of claim 30, wherein the EBV-induced cancer of the lymphocytic origin is an EBV-induced: Burkitt lymphoma, Hodgkin's lymphoma, diffuse large B-cell lymphoma, or a lymphoproliferative disease.

32. The method of claim 30, wherein the EBV-induced cancer of the epithelial origin is an EBV-induced nasopharyngeal carcinoma (NPC) or an EBV-induced gastric cancer/carcinoma.

33. The method of claim 29, wherein the exosomes are obtained from the subject's Vδ2⁺ T cells.

34. The method of claims 29 to 33, wherein the exosomes are obtained from Vδ2⁺ T cells from the subject when the subject was known to be free of cancer.

35. The method of claim 29, wherein the exosomes are obtained Vδ2⁺ T cells from an individual who is allogeneic to the subject.

36. The method of claim 29, comprising administering the exosomes via a route selected from oral, rectal, nasal, topical, buccal, sublingual, transdermal, vaginal, intramuscular, subcutaneous, intravenous, epidural, intrathecal, and central.

37. A composition comprising the exosomes from Vδ2⁺ T cells isolated according to the method of claim 16 and a pharmaceutically acceptable carrier.

38. The composition of claim 37 that is formulated for administration to a subject via a route selected from oral, rectal, nasal, topical, buccal, sublingual, transdermal, vaginal, intramuscular, subcutaneous, intravenous, epidural, intrathecal, and central.
